(19) United States
(12) Patent Application Publication
(10) Pub. No.: US 2023/0383368 A1
(43) Pub. Date: Nov. 30, 2023

(54) A DETECTION METHOD FOR VIRAL REPLICATION

(71) Applicant: Microbio Pty Ltd, Westlake, Queensland (AU)

(72) Inventors: Flavia Huygens, Westlake, QLD (AU); Lisa Simms, Westlake, QLD (AU)

(73) Assignee: Microbio Pty Ltd, Westlake, Queensland (AU)

(21) Appl. No.: 18/018,488

(22) PCT Filed: Jul. 27, 2021
(86) PCT No.: PCT/AU2021/050814

§ 371 Date: Jan. 27, 2023

(30) Foreign Application Priority Data
Jul. 27, 2020 (AU) 2020902624

(51) Int. Cl. C12Q 1/70

(52) U.S. Cl. CPC C12Q 1/701; C12Q 2600/158; C12Q 2600/118; C12Q 1/686

(57) ABSTRACT

The present invention relates to a method for detecting replication of severe acute respiratory syndrome coronavirus 2 (SARS-CoV-2) in a sample, said method including the steps of:
(a) amplifying a target nucleic acid from a portion of subgenomic mRNA of SARS-CoV-2 from the sample; and
(b) detecting the presence or absence of the target nucleic acid amplified in step (a).

### CROSS REFERENCE TO RELATED APPLICATIONS

This is the U.S. National Stage of International Application No. PCT/AU2021/050814, filed Jul. 27, 2021, which was published in English under PCT Article 21(2), which in turn claims the benefit of Australian Patent Application No. 2020902624, filed Jul. 27, 2020, which is incorporated herein in its entirety.

### TECHNICAL FIELD

This invention relates to detection methods of viral replication. More particularly, this invention relates to methods and agents for detecting the replication of severe acute respiratory syndrome coronavirus 2 (SARS-CoV-2) in samples, such as biological samples. The methods and agents are based on the use or detection of subgenomic mRNA specific to SARS-CoV-2, such as by high resolution melt analysis.

### BACKGROUND

Coronavirus disease 2019 (COVID-19) is an infectious viral disease caused by severe acute respiratory syndrome coronavirus 2 (SARS-CoV-2). The first cases of this new viral disease were identified in December 2019 in Wuhan, China, which has now escalated into an ongoing global pandemic.

Current testing for COVID-19 aims to detect the causative virus, SARS-CoV-2, or an immune response to SARS-CoV-2. The two main types of SARS-CoV-2 tests are: nucleic acid detection tests using reverse transcriptase-polymerase chain reaction (RT-PCR) to detect SARS-CoV-2 viral RNA; and serological tests to detect IgM and/or IgG antibodies against the SARS-CoV-2 virus.

Nucleic acid/PCR tests are currently considered to be more clinically sensitive than serology assays for detecting early COVID-19 infections, because they directly detect viral RNA and act as an indicator for viral shedding. The extent to which a positive PCR result correlates with the infectious state of an individual, however, is yet to be determined. This is important as clinical resolution and consecutive negative PCR tests in a previously positive individual are currently being used as criteria when considering a patient's release from isolation.

Existing PCR tests cannot determine whether a person being tested is emitting whole virus capable of infecting someone else, or if the nasopharyngeal swabs instead are collecting viral debris shed after an infection. As a consequence, multiple reports have emerged showing that some people who have recovered from the illness continue to test positive for long periods by PCR, and people who had tested negative twice, and considered cured, then subsequently test positive again.

Currently, the only way to know if a person is still infectious—shedding or emitting replication-competent (“live”) virus—is to culture virus from a specimen belonging to that individual. Culturing (or growing) virus is time-consuming, expensive, requires a high level of biosecurity controls in place, and is impractical for testing/screening purposes.

Without a relatively inexpensive, rapid “live” diagnostic assay available, confusion will remain over who is emitting infectious SARS-CoV-2 virus and who is not. Accordingly, there remains a need for rapid, reliable and cost effective techniques for the detection of SARS-CoV-2 replication in samples, such as biological samples obtained from human subjects potentially infected with the virus.

### SUMMARY

The present invention is predicated in part on the surprising discovery that portions of the subgenomic mRNA of SARS-CoV-2 may be useful in detecting replication of SARS-CoV-2 in a sample, such as a biological sample obtained from a subject infected with SARS-CoV-2.

In a first aspect, the invention provides a method for detecting replication of severe acute respiratory syndrome coronavirus 2 (SARS-CoV-2) in a sample, said method including the steps of:
(a) amplifying a target nucleic acid from a portion of subgenomic mRNA of SARS-CoV-2 from the sample; and
(b) detecting the presence or absence of the target nucleic acid amplified in step (a).

Suitably, the presence or absence of the target nucleic acid is detected at least in part using high resolution melt analysis.

In various embodiments, the present method includes the further step of detecting the presence or absence of SARS-CoV-2 genomic RNA in the sample.

In particular embodiments, the sample is a biological sample, such as cells, blood, serum, plasma, saliva, cerebrospinal fluid, urine, stool, sputum, nasopharyngeal aspirates or swabs, obtained from a subject. In this regard, the subject suitably is or has previously been infected with SARS-CoV-2. Accordingly, the current method may be used for identifying subjects infected with SARS-CoV-2. Additionally, the present method may be used at least in part to determine whether a subject that is presently infected with SARS-CoV-2 remains infectious.

In a second aspect, the invention resides in a method of determining a prognosis for a SARS-CoV-2 infection in a subject:
(a) amplifying a target nucleic acid from a portion of subgenomic mRNA of SARS-CoV-2 from a biological sample from the subject; and
(b) detecting the presence or absence of the target nucleic acid amplified in step (a), wherein the presence of absence of the target nucleic acid indicates a more or less favourable prognosis for the SARS-CoV-2 infection in the subject.

Suitably, step (b) of the present method further includes the step of measuring an expression level of the target nucleic acid. To this end, the expression level of the target nucleic acid can indicate or correlate with a level of replication of SARS-CoV-2 in the biological sample. In some embodiments, the expression level of the target nucleic acid and/or the level of replication indicates or correlates with a more or less favourable prognosis for the SARS-CoV-2 infection in the subject. By way of example, a high or increased expression level of the target nucleic acid and/or level of replication can indicate or correlate with a less favourable prognosis for the SARS-CoV-2 infection and/or a low or decreased expression level of the target nucleic acid and/or level of replication can indicate or correlate with a more favourable prognosis for the SARS-CoV-2 infection.

In a third aspect, the invention relates to a method for identifying an agent capable of altering or modulating replication of SARS-CoV-2 in one or more cells infected therewith, said method including the steps of:
(a) amplifying a target nucleic acid from a portion of subgenomic mRNA of SARS-CoV-2 from the one or more cells treated with the agent; and
(b) measuring an expression level of the target nucleic acid amplified in step (a), wherein the expression level of the target nucleic acid indicates or correlates with a level of replication of SARS-CoV-2 in the one or more cells.

In some embodiments, the present method comprises determining the level of replication of: (i) a first population of the one or more cells treated with the agent; and (ii) a second population of the one or more cells not treated with the agent; and comparing the level of replication of the first and second population of cells, wherein an alteration or modulation of the level of replication determined for the first and second populations of the cells indicates that the agent is capable of altering replication of SARS-CoV-2.

In alternative embodiments, the present method comprises determining the level of replication of the cells at respective first and second time points; and comparing the level of replication of the cells at the first and second time points, wherein an alteration or modulation of the level of replication measured at the first and second time points indicates that the agent is capable of altering replication of SARS-CoV-2.

Suitably, the current method further includes the initial steps of infecting the one or more cells with SARS-CoV-2 and/or treating the one or more cells with the agent.

Referring to the methods of the aforementioned aspects, the portion of subgenomic mRNA suitably comprises at least a portion of a leader sequence.

For the methods of the above aspects, the portion of subgenomic mRNA suitably comprises at least a portion of an E gene or gene product thereof.

In various embodiments of the first, second and third aspects, the portion of subgenomic mRNA of SARS-CoV-2 and/or the target nucleic acid comprises, consists of or consists essentially of a nucleotide sequence set forth in SEQ ID NO:6 or a fragment or variant thereof.

Suitably, for the three aforementioned aspects, the step of amplifying the target nucleic acid includes using one or more primers that hybridize or anneal to (e.g., are complementary or substantially complementary to) the portion of SARS-CoV-2 subgenomic mRNA or a cDNA sequence derived therefrom. In some embodiments, amplifying the target nucleic acid includes using a forward primer that hybridizes to the portion of the leader sequence or a cDNA sequence derived therefrom. In other embodiments, amplifying the target nucleic acid includes using a reverse primer that hybridizes to the portion of the E gene or a cDNA sequence derived therefrom. In specific embodiments, amplifying the target nucleic acid includes using a forward primer comprising, consisting of or consisting essentially of a nucleotide sequence set forth in SEQ ID NO:4, a nucleotide sequence complementary thereto or a fragment or variant thereof and/or a reverse primer comprising, consisting of or consisting essentially of a nucleotide sequence set forth in SEQ ID NO:5, a nucleotide sequence complementary thereto or a fragment or variant thereof.

Suitably, in respect of the above aspects, amplifying the target nucleic acid utilizes reverse transcriptase-polymerase chain reaction (RT-PCR). In this regard, the methods of the invention may further include the steps of obtaining RNA from the sample, the biological sample or one or more cells and reverse transcribing the RNA to obtain complementary DNA (cDNA).

In a fourth aspect, the invention relates to an isolated probe, tool or reagent capable of detecting SARS-CoV-2 in a sample, wherein the probe, tool or reagent is capable of binding, detecting or identifying the presence or absence of subgenomic mRNA of SARS-CoV-2.

In a fifth aspect, the invention provides an isolated oligonucleotide or primer comprising, consisting of or consisting essentially of a nucleic acid sequence as set forth in any one of SEQ ID NOs:4 and 5, a nucleotide sequence complementary thereto or a fragment or variant thereof.

In a sixth aspect, the invention resides in an array comprising the isolated probe, tool or reagent of the fourth aspect and/or the isolated oligonucleotide of the fifth aspect.

In a seventh aspect, the invention relates to a biochip comprising a solid substrate and the isolated probe, tool or reagent of the fourth aspect and/or the isolated oligonucleotide of the fifth aspect.

In an eighth aspect, the invention provides a kit or assay for detecting replication of SARS-CoV-2 in a sample, said kit or assay comprising: the isolated probe, tool or reagent of the fourth aspect, the isolated oligonucleotide of the fifth aspect; the array of the sixth aspect; and/or the biochip of the seventh aspect.

Suitably, the kit provides a pair of oligonucleotides, wherein at least one of the pair of oligonucleotides comprises, consists of or consists essentially of the nucleic acid sequence as set forth in any one of SEQ ID NOs:4 and 5. In some embodiments, the kit further comprises a second pair of primers for detecting the presence or absence of SARS-CoV-2 genomic RNA.

In particular embodiments, the kit includes instructions for use, such as instructions for using the pair of oligonucleotides in RT-PCR and high resolution melt analysis.

Suitably, the isolated probe, tool or reagent of the fourth aspect, the isolated oligonucleotide of the fifth aspect, the array of the sixth aspect, the biochip of the seventh aspect or the kit or assay of the eighth aspect, are for use in the method of the first, second and/or third aspects.

Unless the context requires otherwise, the terms “comprise”, “comprises” and “comprising”, or similar terms are intended to mean a non-exclusive inclusion, such that a recited list of elements or features does not include those stated or listed elements solely, but may include other elements or features that are not listed or stated.

By “consist essentially of” is meant in this context that the nucleic acids described herein have one, two or no more than three nucleic acid residues in addition to the recited nucleic acid sequence. The additional nucleic acid residues may occur at the 5′ and/or 3′ ends of the recited nucleic acid sequence, although without limitation thereto.

The indefinite articles ‘a’ and ‘an’ are used herein to refer to or encompass singular or plural elements or features and should not be taken as meaning or defining “one” or a “single” element or feature. For example, “a” cell includes one cell, one or more cells and a plurality of cells.

As generally used herein “about” refers to a tolerance or variation in a stated value or amount that does not appreciably or substantially affect function, activity or efficacy. Typically, the tolerance or variation is no more than 10%, 5%, 3%, 2%, or 1% above or below a stated value or amount.

The reference to any prior art in this specification is not, and should not be taken as an acknowledgement or any form of suggestion that the prior art forms part of the common general knowledge.

### BRIEF DESCRIPTION OF THE FIGURES

FIGS. 1A-1F. Whole genome of Wuhan SARS-CoV-2 strain. SEQ ID NO: 1 is shown.

FIG. 2. An exemplary amplicon of subgenomic mRNA from SARS-CoV-2 for use in detecting viral replication. The sequence shows Leader+E gene sequence by discontinuous transcription. 5′ leader is joined to an mRNA “body” including TRS (bold) sequence. Total length of sgRNA amplicon=171 bp. Forward and Reverse primer locations in italics. Transcription regulating sequence (TRS) in bold text. E gene in underline text. SEQ ID NO: 6 is shown.

FIG. 3. RT-PCR results for replicating SARS-CoV-2 test using tenfold serial dilution of SARS-CoV-2 total RNA extracted from cell culture. Normalised fluorescence is shown on the Y axis and number of cycles on the X axis.

FIG. 4. High Resolution Melt curve results for replicating SARS-CoV-2 test using tenfold serial dilution of SARS-CoV-2 total RNA extracted from cell culture. The change in fluorescence (dF/dT) is shown on the Y axis and temperature is shown on the X axis.

FIG. 5. Exemplary negative and positive HRM curves for the detection of replication of SARS-CoV-2:
(a) shows the results when:
   Internal control: NEGATIVE
   Valid PCR: no*
   CoV-2: no result
   CoV-2 Live virus present: no result

Possible contribution factors to the failure of the procedure are poor RNA integrity, RNA input concentration outside recommended range and/or presence of PCR inhibitors in sample;
(b) shows the results when:
   Internal control: POSITIVE
   Valid PCR: yes
   CoV-2: NEGATIVE
   CoV-2 Live virus present: NEGATIVE;
(c) shows the results when:
   Internal control: POSITIVE
   Valid PCR: yes
   CoV-2: POSITIVE
   CoV-2 Live virus present: NEGATIVE;
(d) shows the results when:
   Internal control: POSITIVE
   Valid PCR: yes
   CoV-2: POSITIVE
   CoV-2 Live virus present: POSITIVE

FIG. 6. Instrument panel showing run profile conditions for RT-PCR assay.

FIG. 7. Melt curve for Bio-Rad CFX96 real-time PCR machine.

FIG. 8. Melt curve for MIC (Biomolecular Systems) real-time PCR machine.

FIG. 9. Melt curve for QuantStudio 5 (Applied Biosystems) real-time PCR machine.

FIG. 10. Melt curve for RotorGeneQ (Qiagen) real-time PCR machine.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named “Sequence.txt” (28,672 bytes), which was created on Jan. 26, 2023, which is incorporated by reference herein.

### BRIEF DESCRIPTION OF THE SEQUENCES

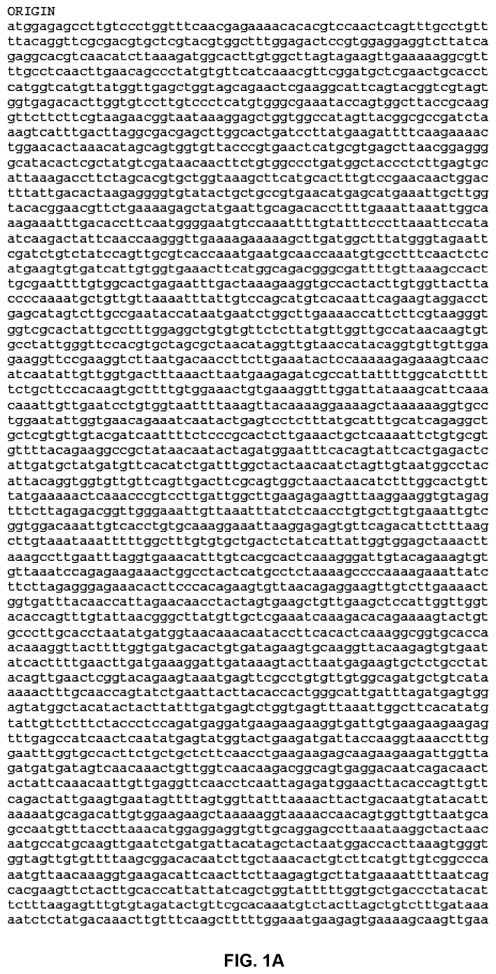

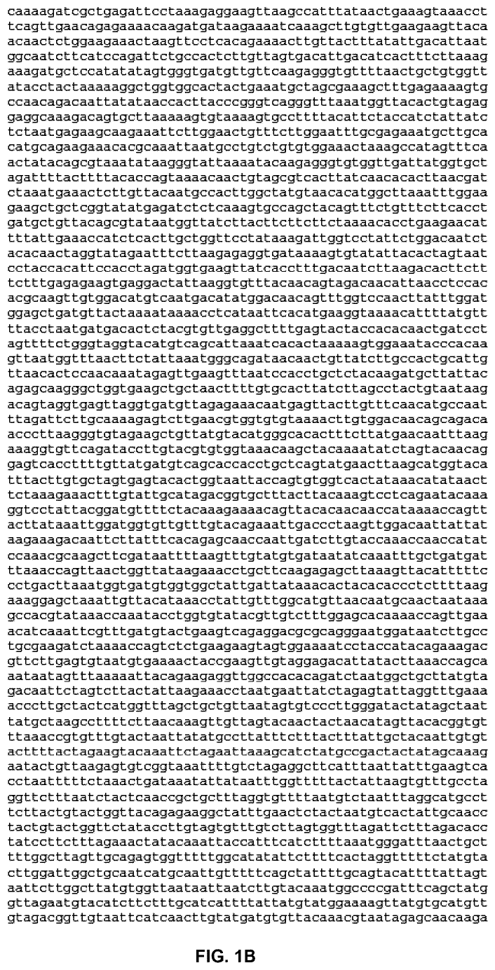

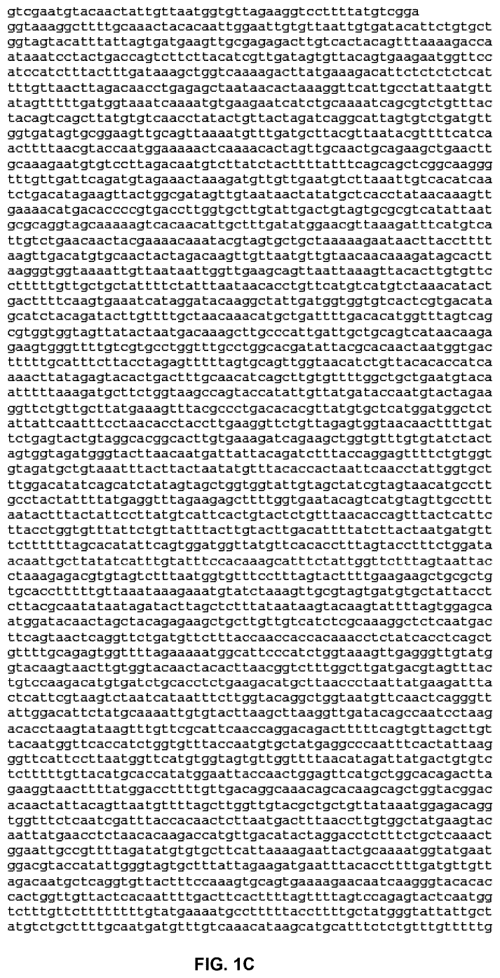

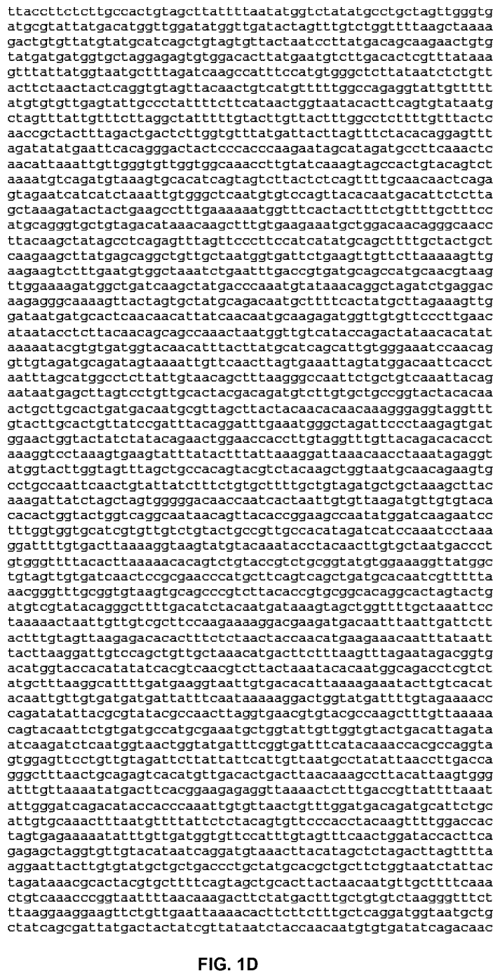

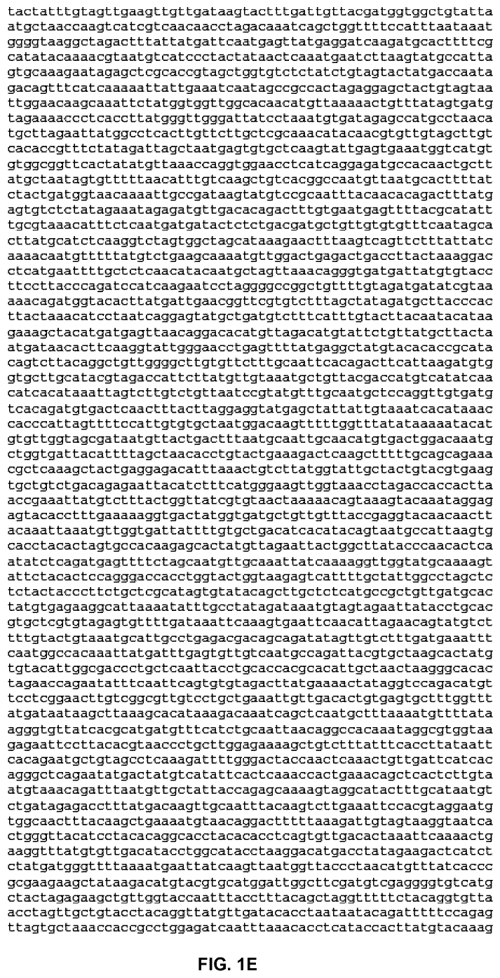

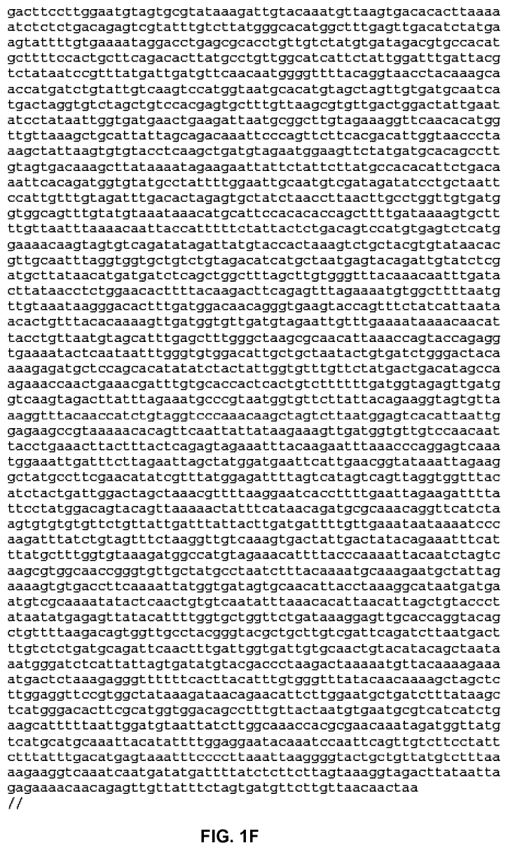

SEQ ID NO:1 Whole genome of Wuhan SARS-CoV-2 strain in FIGS. 1A-1F

SEQ ID NO:2 Forward internal control primer (IC_RPP30-F) in Table 1

SEQ ID NO:3 Reverse internal control primer (IC_RPP30-R) in Table 1

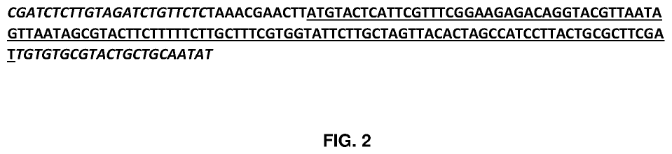

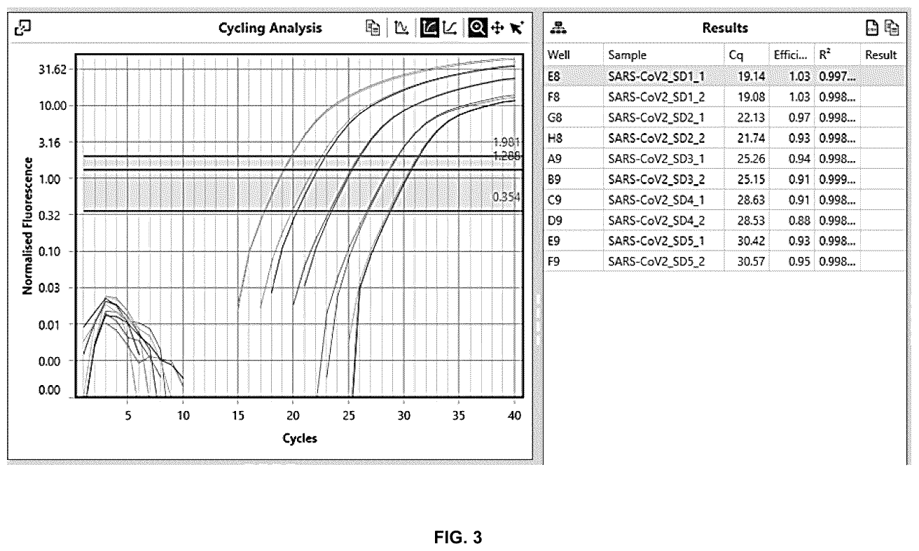

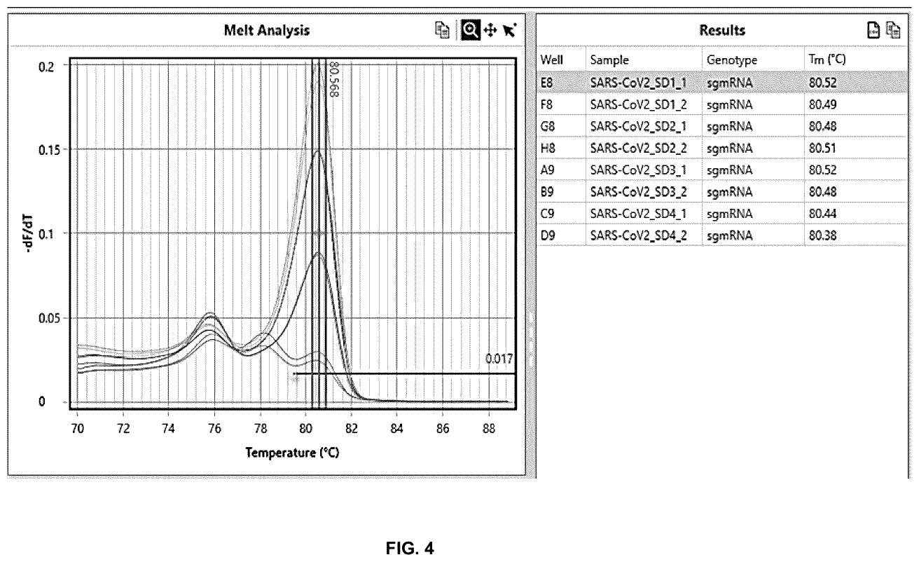

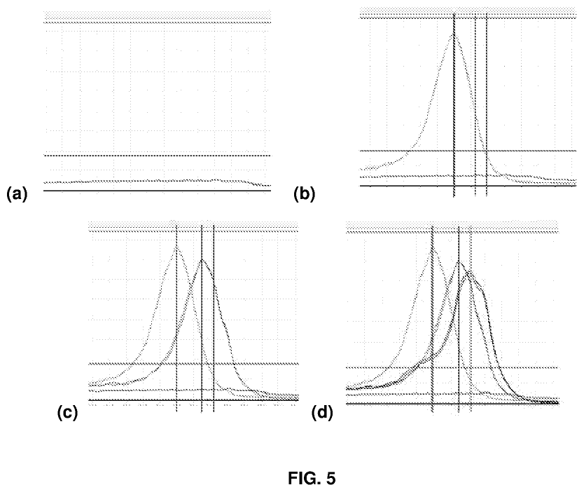

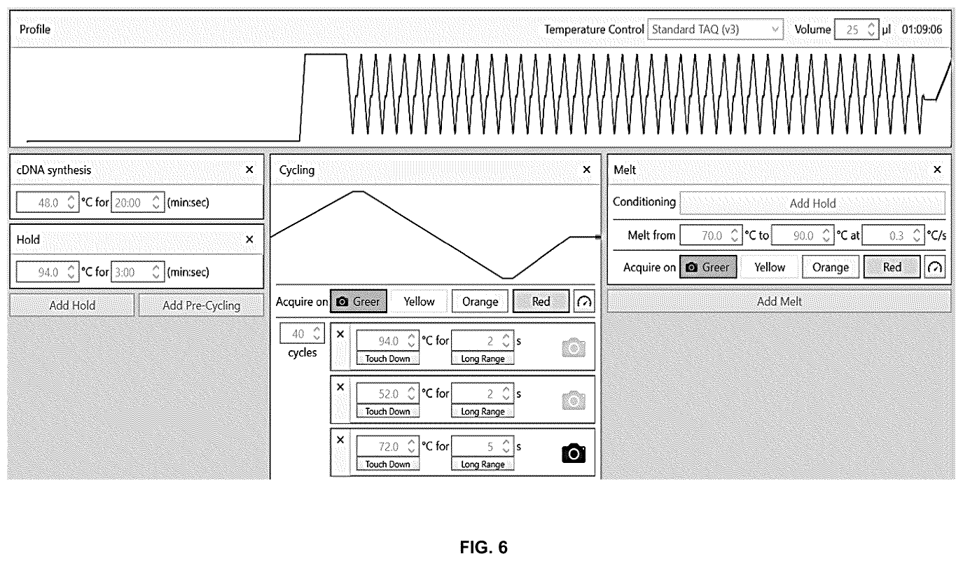

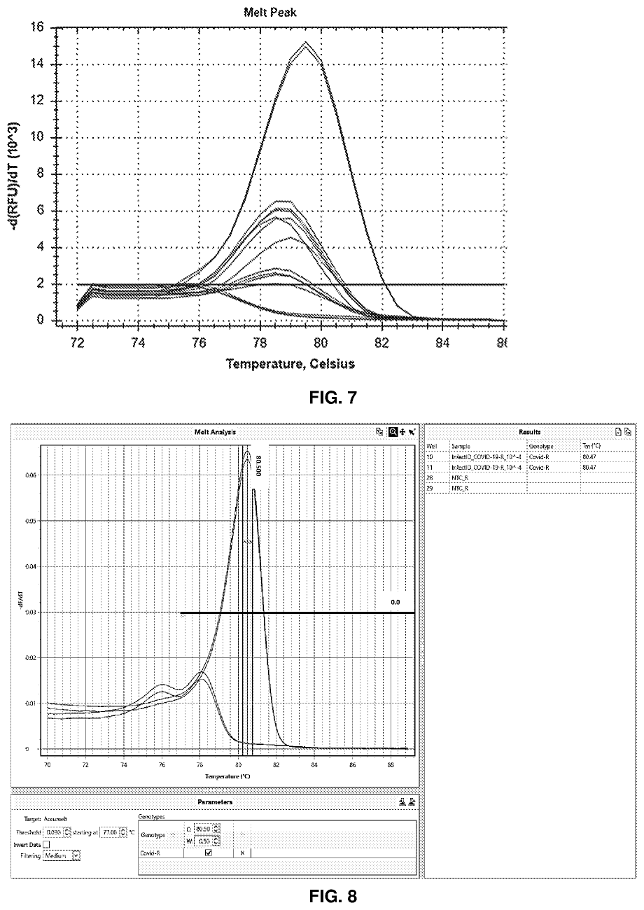

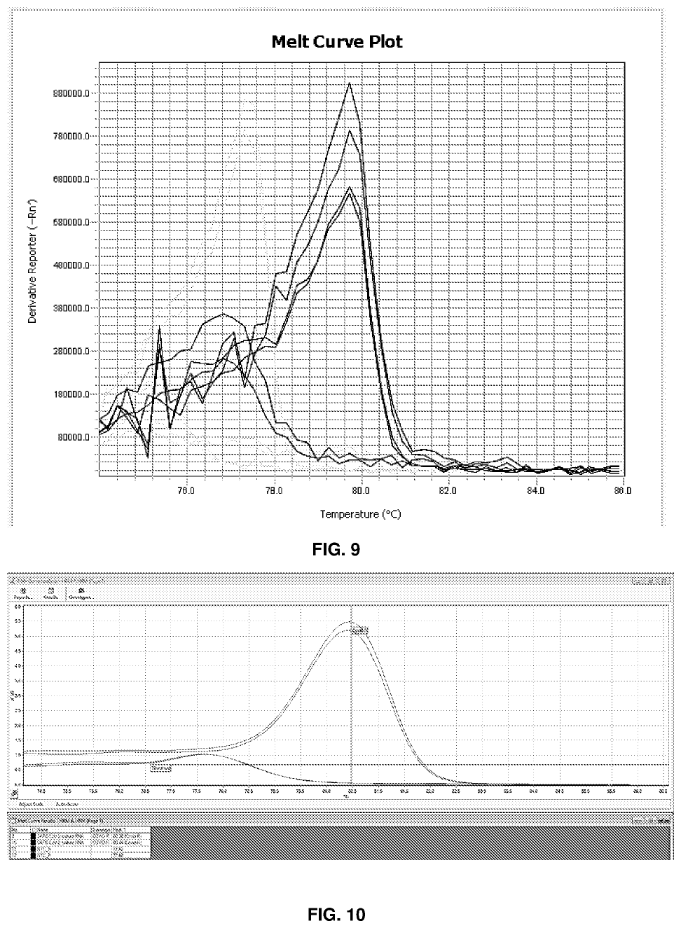

SEQ ID NO:4 Forward SARS-CoV-2 sgRNA primer (sgRNA-F) in Table 1 and FIG. 2

SEQ ID NO:5 Reverse SARS-CoV-2 sgRNA primer (sgRNA-R) in Table 1 and FIG. 2

SEQ ID NO:6 Target nucleic acid amplicon of sgRNA of SARS-CoV-2 in FIG. 2

### DETAILED DESCRIPTION

The present invention is predicated on the surprising discovery that subgenomic mRNA specific to SARS-CoV-2 can be utilised as an indicator of replication of this coronavirus in samples, such as biological samples obtained from human subjects. Advantageously, detecting the presence of such subgenomic mRNA of SARS-CoV-2 can advantageously be used to indicate patients that are presently infectious with the virus, which is unlike those PCR tests for SARS-CoV-2 that test for the presence of genomic RNA of the virus.

In a broad form, the method includes the step of detecting the presence or absence of subgenomic mRNA from SARS-CoV-2 in a sample.

Accordingly, in one aspect, the invention provides a method for detecting replication of SARS-CoV-2 in a sample, said method including the steps of:
(a) amplifying a target nucleic acid from a portion of subgenomic mRNA of SARS-CoV-2 from the sample; and
(b) detecting the presence or absence of the target nucleic acid amplified in step (a).

Coronaviruses (order Nidovirales, family Coronaviridae) represent a diverse group of enveloped, positive-stranded RNA viruses. The coronavirus genome, approximately 27-32 Kb in length, is the largest found in any of the RNA viruses. Large Spike (S) glycoproteins protrude from the virus particle giving coronaviruses a distinctive corona-like appearance when visualized by electron microscopy. Coronaviruses infect a wide variety of species, including canine, feline, porcine, murine, bovine, avian and human (Holmes, et al., 1996. Coronaviridae: the viruses and their replication, p. 1075-1094. In Fields (ed.), Fields Virology. Lippincott-Raven, Philadelphia, Pa.).

The terms “severe acute respiratory syndrome coronavirus 2” and “SARS-CoV-2” refer to the coronavirus that is widely recognized as the etiologic agent of the COVID-19 pandemic that was first identified in Wuhan of the Hubei province of China. It is envisaged that the term encompasses all isolates and strains of SARS-CoV-2 as are known in the art, inclusive of the “L” and “S” strains. An exemplary genome of SARS-CoV-2 is provided in FIGS. 1A-1F.

The term “replication”, as it relates to SARS-CoV-2, includes, but is not limited to, the steps of adsorbing (e.g., receptor binding) to a cell, entry into a cell (such as by endocytosis), introducing its genome sequence into the cell, uncoating the viral genome, initiating transcription of SARS-CoV-2 genomic RNA to produce subgenomic mRNA, directing expression of SARS-CoV-2 encapsidation proteins and/or encapsidation of the replicated viral nucleic acid sequence with the encapsidation proteins into a viral particle that is released from the cell to infect other cells that are of either a permissive or susceptible character.

The term “nucleic acid” as used herein designates single- or double-stranded mRNA, RNA, cRNA, RNAi, siRNA and DNA inclusive of cDNA, mitochondrial DNA (mtDNA) and genomic DNA. The present invention also contemplates nucleic acids that have been modified such as by taking advantage of codon sequence redundancy. In a more particular example, codon usage may be modified to optimize expression of a nucleic acid in a particular organism or cell type. The invention further provides use of modified purines (for example, inosine, methylinosine and methyladenosine) and modified pyrimidines (for example, thiouridine and methylcytosine) in isolated nucleic acids of the invention.

The nucleotide symbols are set forth in the following table:

Nucleotide Symbols

| Symbol | Description |
|---|---|
| A | Adenosine |
| C | Cytidine |
| G | Guanosine |
| T | Thymidine |
| U | Uridine |
| M | Amino (adenosine, cytosine) |
| K | Keto (guanosine, thymidine) |
| R | Purine (adenosine, guanosine) |
| Y | Pyrimidine (cytosine, thymidine) |
| N | Any nucleotide |

The terms “subgenomic mRNA” and “sgRNA” are used interchangeably herein to refer to an RNA molecule of a length or size which is smaller than the genomic RNA from which it was derived (i.e., a partial genomic sequence). The subgenomic mRNA can be transcribed from an internal promoter, whose sequences reside within the genomic RNA or its complement. Transcription of the subgenomic mRNA may be mediated by viral-encoded polymerase(s) associated with host cell-encoded proteins, ribonucleoprotein(s), or a combination thereof. An exemplary portion of subgenomic mRNA of SARS-CoV-2 is set forth in SEQ ID NO:6 in FIG. 2.

Generally, subgenomic mRNA can include at least a portion of a leader sequence. The term “leader sequence” refers to a sequence of about 40 to about 150, about 50 to about 80, and or about 55 to about 75 nucleotides that is located at the 5′ terminus of a viral genome. This sequence is juxtaposed to the 5′ terminus of each subgenomic mRNA by transcriptional mechanisms during synthesis. The leader sequence typically plays a role in the generation of the subgenomic mRNA transcripts, transcription, replication, translation and/or packaging of viral RNA.

In some embodiments described herein, the portion of subgenomic mRNA of SARS-CoV-2 suitably comprises at least a portion of an E gene or gene product thereof. As will be appreciated by the skilled artisan, the E gene encodes the envelope or E protein of coronaviruses. The coronavirus E protein has a well-established role in the assembly of virions where it may induce membrane curvature or aid in membrane scission.

By “gene” is meant a unit of inheritance that occupies a specific locus on a genome and consists of transcriptional and/or translational regulatory sequences and/or a coding region and/or non-translated sequences (i.e., introns, 5′ and 3′ untranslated sequences). By “gene product” is meant a product of the recited gene. By way of example, gene products can include protein and cDNA sequences derived from viral RNA gene sequences, such as the leader sequence or the E gene.

In particular embodiments, the target nucleic acid is amplified at least partly from a conserved region of subgenomic mRNA of SARS-CoV-2. The terms “conserved region” or “conserved sequence” as used herein refer to a segment of nucleotide sequence of a gene or amino acid sequence of a protein that is significantly similar between various different nucleotide sequences of the particular gene or protein, such as that of different coronavirus strains or isolates. It is envisaged that the conserved region of the subgenomic mRNA of SARS-CoV-2 can be determined by any means or method known in the art.

In various embodiments of the methods described herein, the target nucleic acid comprises, consists of or consists essentially of a nucleotide sequence set forth in SEQ ID NO:6 or a fragment or variant thereof.

In various embodiments, the present method includes the further step of detecting the presence or absence of SARS-CoV-2 genomic RNA (gRNA) in the sample, such as by amplifying a further nucleic acid from a portion of genomic RNA specific to SARS-CoV-2 and subsequently detecting the further nucleic acid. To this end, the present method may comprise an assay, such as a multiplex assay, for detecting genomic RNA (gRNA) and subgenomic mRNA (sgRNA) from SARS-CoV-2, as shown in FIG. 5. Exemplary methods, including primers and probes, for detecting the presence or absence of SARS-CoV-2 genomic RNA are described in Udugama et al. ACS Nano 2020, 14, 4, 3822-3835 and Carter et al. ACS Cent. Sci. 2020, 6, 5, 591-605.

The target nucleic acid, such as a portion of subgenomic mRNA of SARS-CoV-2, that is analysed or amplified according to the methods of the present invention may be analysed while within the sample (e.g., directly amplified within the sample), or may first be extracted, isolated or purified from the sample (e.g., within genetic material isolated from the sample prior to amplification). Any method for isolating nucleic acid, such as RNA or total RNA, from a sample can be used in the methods of the present invention, and such methods are well known to those of skill in the art. The extracted nucleic acid can include DNA and/or RNA (including single stranded viral RNA). It is envisaged that the particular steps and/or techniques of isolating a sample, such as a biological sample from a subject, processing a sample and nucleic acid extraction, detection and characterisation (e.g., RNA extraction, detection and characterisation), can be carried out in any suitable way known in the art.

As used herein, by “isolated” is meant material, such as genetic material or a probe, tool, reagent, oligonucleotide or primer, that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in recombinant, chemical synthetic, enriched, purified or partially purified form.

The methods described herein include amplification of the target nucleic acid. By way of example, the methods can include amplifying a nucleic acid of the subgenomic mRNA of SARS-CoV-2 from genetic material obtained from the sample, the biological samples or the cells to form an amplification product. “Amplification product” or “amplicon” refers to a nucleic acid product generated by nucleic acid amplification techniques. The nucleic acid may be amplified by any method known in the art including, but not limited to polymerase chain reaction (PCR), ligase chain reaction (LCR) and reverse transcription-polymerase chain reaction (RT-PCR) using one or more oligonucleotides/primers that will amplify transcribed RNA. To this end, a further step of reverse transcription can be included in the methods of the invention prior to analysis. Thus, the target nucleic acid to be amplified can include one or more portions of subgenomic mRNA, such as portions of the leader sequence and E gene, a cDNA copy thereof or any combination thereof.

Suitable nucleic acid amplification techniques are well known to a person or ordinary skill in the art, and include polymerase chain reaction (PCR) as for example described in Ausubel et al., Current Protocols in Molecular Biology (John Wiley & Sons, Inc. 1994-1998, strand displacement amplification (SDA) as for example described in U.S. Pat. No. 5,422,252, rolling circle replication (RCR) as, for example, described in Liu et al, 1996, J. Am. Chem. Soc. 118: 1587-1594 and in WO 92/01813 and WO 97/19193, nucleic acid sequence-based amplification (NASBA) as for example described in Sooknanan et al., 1994, Biotechniques 17:1077-1080, ligase chain reaction (LCR), simple sequence repeat analysis (SSR), branched DNA amplification assay (b-DNA), transcription amplification and self-sustained sequence replication, and Q-β replicase amplification as for example described in Tyagi et al., 1996, Proc. Natl. Acad. Sci. USA 93:5395-5400.

Given the single stranded RNA (ssRNA) nature of the subgenomic mRNA of SARS-CoV-2, amplifying the target nucleic acid of the subgenomic mRNA or gene product thereof suitably utilizes reverse transcriptase-polymerase chain reaction (RT-PCR) to generate a corresponding cDNA template of the subgenomic mRNA suitable for amplification by standard PCR techniques, such as those disclosed herein. To this end, the present method may further include the steps of obtaining or isolating RNA, such as total RNA, from the sample in question and reverse transcribing the isolated RNA to obtain cDNA.

Such amplification methods can utilise one or more oligonucleotide primers, including, for example, an amplification primer pair, such as those provided in SEQ ID NOs: 4 and 5, that selectively hybridize or anneal to either end of the target nucleic acid. Oligonucleotide primers useful in practicing the methods of the invention can include, for example, an oligonucleotide that is complementary to and spans a portion of the target nucleic acid that is a conserved region and/or specific to SARS-CoV-2. In this regard, one or both primers may span or include the position of one or more SNPs or polymorphic regions specific to SARS-CoV-2, such that the presence of subgenomic mRNA of SARS-CoV-2 is detected by the presence or absence of selective amplification of the target nucleic acid. The basis for this is that primers or oligonucleotides with one or more mismatched residues will not function as primers in a PCR under appropriate conditions. As a result, if nucleic acid having a given SNP or polymorphic region in subgenomic mRNA of SARS-CoV-2 is present in the sample, the primer pair specific or complementary to that SNP or polymorphic region will produce an amplification product but not to nucleic acid from or specific to another virus, such as other coronavirus strains or isolates, that do not possess the specific SNP(s) or polymorphic region(s) in their subgenomic mRNA. Accordingly, the methods described herein can also facilitate in the narrowing down or, in some cases, confirming the detection and/or replication of SARS-CoV-2 over another coronavirus strain or isolate in the sample, the biological sample or the one or more cells.

As used herein, the term “single nucleotide polymorphism” (SNP) refers to nucleotide sequence variations that occur when a single nucleotide (A, T, C or G) in the genome sequenceis altered (such as via substitutions, addition or deletion). SNPs can occur in both coding (gene) and noncoding regions of the genome such as the genome of a prokaryotic or eukaryotic microorganism.

Suitably, for the methods described herein, the step of amplifying the nucleic acid includes using one or more primers that hybridize or anneal to (e.g., are complementary or substantially complementary to) to respective portions or ends of the target nucleic acid of the portion of SARS-CoV-2 subgenomic mRNA or a cDNA sequence derived therefrom. In specific embodiments, a primer of the invention hybridizes to or is complementary to a portion of subgenomic mRNA of SARS-CoV-2 of the nucleic acid sequence set forth in SEQ ID NO: 6 or a gene product thereof (e.g., cDNA).

In some embodiments, amplifying the target nucleic acid includes using a forward primer that hybridizes to the portion of the leader sequence or a cDNA sequence derived therefrom, such as that provided herein. In other embodiments, amplifying the target nucleic acid includes using a reverse primer that hybridizes to the portion of the E gene or a cDNA sequence derived therefrom, such as that disclosed herein.

Exemplary primer sequences are provided in SEQ ID NOs:2 to 5 shown in Table 1. In specific embodiments, amplifying the target nucleic acid includes using a forward primer comprising, consisting of or consisting essentially of a nucleotide sequence set forth in SEQ ID NO:4, a nucleotide sequence complementary thereto or a fragment or variant thereof and/or a reverse primer comprising, consisting of or consisting essentially of a nucleotide sequence set forth in SEQ ID NO:5, a nucleotide sequence complementary thereto or a fragment or variant thereof.

By “primer” it is meant an oligonucleotide which, when paired with a strand of DNA, is capable of initiating the synthesis of a primer extension product in the presence of a suitable polymerizing agent, such as a DNA polymerase (e.g., Taq polymerase, RNA-dependent DNA polymerase, Sequenase™). The primer is suitably single-stranded for maximum efficiency in amplification but can alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the oligonucleotide primer typically contains 10 to 35 or more nucleotide residues (e.g., about 10, 11, 12, 13, 14, 15, 16, 17, 18, 19, 20, 21, 22, 23, 24, 25, 26, 27, 28, 29, 30, 31, 32, 33, 34, 35 nucleotides in length including any range therein), although it can contain fewer nucleotide residues. Primers can be large polynucleotides, such as from about 200 nucleotides to several kilobases or more. Suitably, the GC ratio of a primer should be above 30%, 35%, 40%, 45%, 50%, 55%, or 60% so as to prevent hair-pin structures forming on the primer. Furthermore, the amplicon or nucleic acid to be amplified should be sufficiently long enough to be detected by standard molecular biology methodologies. Suitably, the amplicon or amplified nucleic acid is at least about 20, 25, 30, 35, 40, 45, 50, 55, 60, 65, 70, 75, 80, 85, 90, 95, 100, 110, 120, 130, 140, 150, 175, 200, 250, 300, 350, 400, 450, 500, 550, 600, 700, 800, or 1000 base pairs in length.

Primers can be selected to be “substantially complementary” to the sequence on the template to which it is designed to hybridize and serve as a site for the initiation of synthesis. By “substantially complementary” it is meant that the primer is sufficiently complementary to hybridize with a target polynucleotide. In some embodiments, the primer contains no mismatches with the template to which it is designed to hybridize with but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

Primers may be manufactured using any convenient method of synthesis. Examples of such methods may be found in “Protocols for Oligonucleotides and Analogues; Synthesis and Properties”, Methods in Molecular Biology Series, Volume 20, Ed. Sudhir Agrawal, Humana ISBN: 0-89603-247-7, 1993. The primers may also be labelled to facilitate detection.

As used herein, “variant” nucleic acids refer to nucleic acids that comprise nucleotide sequences of naturally occurring (e.g., allelic) variants and orthologs (e.g., from a different strain or isolate) of SARS-CoV-2. Preferably, nucleic acid variants share at least 70% or 75%, preferably at least 80% or 85% or more preferably at least 90%, 91%, 92%, 93%, 94%, 95%, 96%, 97%, 98% or 99% sequence identity with a nucleotide sequence disclosed herein.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which identical nucleic acid base (e.g., A, T, C, G) occurs in both sequence to yield the number of matched positions, dividing the number of matched positions by the total number of positions in the window of comparison (i.e., the window size), and multiplying the result by 100 to yield the percentage of sequence identity (% seq. identity).

“Homology” refers to the percentage number of nucleic acids or amino acids that are identical or constitute conservative substitutions. Homology can be determined using sequence comparison programs such as GAP (Deveraux et al., 1984, Nucleic Acids Research 12, 387-395), which is incorporated herein by reference. In this way, sequences of a similar or substantially different length to those cited herein could be compared by insertion of gaps into the alignment, such gaps being determined, for example, by the comparison algorithm used by GAP.

Also included are nucleic acid fragments. A “fragment” is a segment, domain, portion or region of a nucleic acid, which respectively constitutes less than 100% of the nucleotide sequence. In particular embodiments, a nucleic acid fragment may comprise, for example, at least 5, 6, 7, 8, 9, 10, 11, 12, 13, 14, 15, 16, 17, 18, 19, 20, 21, 25, 3035, 40, 45, 50, 55, 60, 65, 70, 75, 80, 85, 90, 95, 100, 125, 150, 175, 200, 225, 250, 275, 300, 325, 350, 375, 400, 425, 450, 475, 500, 600, 700 and 800, 900, 1000, 1500 and 2000 contiguous nucleotides of said nucleic acid.

Generally speaking, the skilled person would appreciate that the step of detecting the presence or absence of the nucleic acid amplified in step (a) can be performed by any means or method known in the art, inclusive of those provided herein.

In particular embodiments, the presence or absence of the nucleic acid is detected at least in part using high resolution melt (HRM) analysis. HRM analysis advantageously allows for the simple (e.g., no use of labelled probes), rapid (e.g., less than 45 minutes) and reliable detection of SARS-CoV-2 in samples. With the portability of some newer platforms, such as the Mic qPCR cycler from Bio Molecular Systems, PCR-based amplification and HRM analysis of a target nucleic acid, and hence SARS-CoV-2 detection, can also be performed remotely or at point of care (POC) facilities, precluding the need for transport of samples to, for example, external testing laboratories.

HRM is based upon the accurate monitoring of changes in fluorescence as a PCR product (i.e., amplicon) stained with an intercalating fluorescent dye is heated through its melting temperature (Tₘ). In contrast to traditional melting, the information in HRM analysis is contained in the shape of the melting curve, rather than just the calculated Tₘ, so HRM may be considered a form of spectroscopy. HRM analysis is a single step and closed tube method, the amplification and melting can be run as a single protocol on a real-time PCR machine.

In embodiments of the present invention, the methods utilise an amplification primer pair that selectively hybridize to a target nucleic acid that is specific to a portion of subgenomic mRNA of SARS-CoV-2, as described herein. The amplification reaction mixture contains the fluorescent dye, which is incorporated into the resulting amplicon. The resulting amplicon is then subjected to HRM with incremental increases in temperature (i.e., 0.01-0.5° C.) ranging from about 50° C. to about 95° C. At some point during this process, the melting temperature of the amplicon is reached and the two strands of DNA separate or “melt” apart.

The HRM is monitored in real-time using the fluorescent dye incorporated into the amplicon. The level of fluorescence of the dye is monitored as the temperature increases with the fluorescence reducing as the amount of double stranded DNA reduces. Changes in fluorescence and temperature can be plotted in a graph known as a melt curve.

As a skilled addressee will understand, the Tₘ of the amplicon at which the two DNA strands separate is predictable, being dependent on the sequence of the nucleotide bases forming the amplicon. Accordingly, it is possible to differentiate between amplicons including an amplicon containing a polymorphism (i.e., a SNP or SNPs) as the melt curves will appear different. Indeed, in some embodiments, it is possible to differentiate between amplicons containing the same polymorphism based on differences in the surrounding DNA sequences.

HRM curves can be discriminated from one another by many different strategies. For example, in many cases, HRM curves can be discriminated on the basis of obvious differences in curve shape and/or on the basis of Tₘ with a difference of 0.2° C. being regarded as significant. In other cases, a difference graph analysis can be used in which a defined curve is used as a baseline with other normalised curves being plotted in relation to the baseline (see Price, E. P., et al., 2007, Appln. Environ. Microbiol, 72:7793-7803). In yet other cases, a difference graph-based method can be used involving deriving the 3rd and 97th centiles from the mean±1.96 standard deviations for the fluorescence at every temperature (see Andersson, P., et al., 2009, Antimicrob. Agents Chemother. 53:2679-2683 and Merchant-Patel, S., et al., 2008, Int. J. Food Microbiol., 128:304-308).

For the methods of the invention described herein, the mere presence or absence of a suitable melt curve, such as shown in FIG. 5, after initial amplification steps with SARS-CoV-2 specific primers can be sufficient to detect replication of SARS-CoV-2 within the sample (e.g., detect the presence of the target nucleic acid amplified from subgenomic mRNA (or cDNA thereof) of SARS-CoV-2). In particular embodiments, however, the melt curve produced from the sample may be compared to a melt curve produced from a positive control nucleic acid (e.g., RNA or cDNA isolated from a SARS-CoV-2 strain or isolate).

In particular embodiments, the methods described herein utilise a probe, such as to detect the presence or absence of an amplified nucleic acid molecule (e.g., the target nucleic acid). “Probe” refers to a molecule that binds to a specific sequence or sub-sequence or other moiety of another molecule. Unless otherwise indicated, the term “probe” typically refers to apolynucleotide probe that binds to another polynucleotide, often called the “target polynucleotide”, through complementary base pairing. Probes can bind target polynucleotides lacking complete sequence complementarity with the probe, depending on the stringency of the hybridization conditions. Probes can be labelled directly or indirectly.

In alternative embodiments, however, the methods of the invention can be performed without the use of a probe, inclusive of labelled probes, such as in those embodiments that utilise HRM.

It will be well appreciated by a person of skill in the art that the isolated nucleic acids, such as isolated oligonucleotides and primers, of the invention can be conveniently prepared using standard protocols such as those described in Chapter 2 and Chapter 3 of CURRENT PROTOCOLS IN MOLECULAR BIOLOGY (Eds. Ausubel et al. John Wiley & Sons NY, 1995-2008).

In some embodiments, complementary nucleic acids hybridise to nucleic acids of the invention under high stringency conditions.

“Hybridise and Hybridisation” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing. In DNA, A pairs with T and C pairs with G. In RNA, U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently.

“Stringency” as used herein, refers to temperature and ionic strength conditions, and presence or absence of certain organic solvents and/or detergents during hybridisation. The higher the stringency, the higher will be the required level of complementarity between hybridizing nucleotide sequences.

“Stringent conditions” designates those conditions under which only nucleic acid having a high frequency of complementary bases will hybridize.

Stringent conditions are well-known in the art, such as described in Chapters 2.9 and 2.10 of Ausubel et al., supra, which are herein incorporated by reference. A skilled addressee will also recognize that various factors can be manipulated to optimize the specificity of the hybridization. Optimization of the stringency of the final washes can serve to ensure a high degree of hybridization.

Suitably, detecting the presence of the target nucleic acid indicates replication of SARS-CoV-2 in the sample. It is further envisaged that the step of detecting the presence or absence of subgenomic mRNA of SARS-CoV-2 could include detecting an expression level thereof. Such an expression level may subsequently used to determine a level of replication of the SARS-CoV-2 virus in the sample, such as hereinafter described.

The sample described herein may be or comprise an environmental sample, such as an air, soil or water sample, a filtrate, a food or manufactured product, or swab from a surface, such as from a medical instrument or workplace surface. In other examples, the sample is obtained from cultured cells.

In various embodiments, the sample referred to herein is a biological sample obtained from a subject. The term “biological sample” as used herein refers to a sample that may be extracted, untreated, treated, diluted or concentrated from a patient or subject. Suitably, the biological sample is selected from any part of a patient or subject's body, including, but not limited to, cells, hair, skin, nails, tissues or bodily fluids, secretions or excretions, such as sputum, saliva, cerebrospinal fluid, serum, plasma, stool, urine and blood. In particular embodiments, the sample is, comprises or is obtained from a nasopharyngeal aspirate or swab.

In further embodiments, the methods described herein further include the initial step of obtaining the sample, such as obtaining the biological sample from the subject in question.

Accordingly, the aforementioned methods may be used for identifying or diagnosing subjects or patients that are currently infected with SARS-CoV-2. Additionally, the present method may be used at least in part to determine whether a subject that is presently infected with SARS-CoV-2 is also infectious (e.g., actively shedding virus particles). To this end, the methods of the present invention are particularly useful in assisting clinicians in determining whether the subject has an active SARS-CoV-2 infection and if so, an appropriate course of action or treatment based on this diagnosis. In particular, the present method can facilitate the identification of subjects in the early stage of a SARS-CoV-2 infection in which the replication-competent (“live”) viral load of SARS-CoV-2 is high and the non-replication-competent (“dead”) viral load of SARS-CoV-2 is low. Current PCR-based detection tests specific for genomic RNA of SARS-CoV-2 often return a negative result in this early infectious period of an infection. To this end, the present method can be used to more accurately define the stage or time when viral shedding of SARS-CoV-2 in an infected subject stops and recovery from the illness begins. These results can then be used by health authorities to determine whether an infected subject is safe to return to, for example, work, sport and/or community activities. Further, the results from the present method can inform border security authorities about, for example, who is safe to cross state borders or enter a country. Accordingly, the current method can advantageously reduce periods of mandatory isolation or quarantine, particularly for those people who are currently infected, or who have come in contact with infected persons, or who have recently returned from overseas.

As used herein, the terms “subject” and “patient” are used interchangeably. However, it will be understood that “patient” does not imply that symptoms are present. As used herein, the terms “subject” and “subjects” refer to an animal, preferably a mammal including a non-primate (e.g., cows, pigs, horses, goats, sheep, cats, dogs, avian species and rodents) and a primate (e.g., monkeys such as a cynomolgous monkey and humans), and more particularly a human.

In a further aspect, the invention resides in a method of determining a prognosis for a SARS-CoV-2 infection in a subject, said method including the steps of:
(a) amplifying a target nucleic acid from a portion of subgenomic mRNA of SARS-CoV-2 from a biological sample from the subject; and
(b) detecting the presence or absence of the target nucleic acid amplified in step (a), wherein the presence of absence of the target nucleic acid indicates a more or less favourable prognosis for the SARS-CoV-2 infection in the subject.

The terms “prognosis” and “prognostic” are used herein to include making a prognosis, which can provide for predicting a clinical outcome (with or without medical treatment), selecting an appropriate course of treatment (or whether treatment would be effective) and/or monitoring a current treatment and potentially changing the treatment. This may be at least partly based on determining expression levels of the target nucleic, which may be in combination with or addition to determining the expression levels of additional protein and/or other nucleic acid biomarkers, as are known in the art. A prognosis may also include a prediction, forecast or anticipation of any lasting or permanent physical or psychological effects of a SARS-CoV-2 infection suffered by the subject after the infection has been successfully treated or otherwise resolved. Furthermore, prognosis may include one or more of determining disease progression (e.g., hospitalisation, intubation and manual ventilation), determining the infectiousness of a patient, determining a stage of SARS-CoV-2 infection, therapeutic responsiveness, implementing appropriate treatment regimes, and determining the probability, likelihood or potential for infection recurrence after therapy. It would be appreciated that a positive prognosis typically refers to a beneficial clinical outcome or outlook, such as little or no clinical symptoms associated with the SARS-CoV-2 infection, whereas a negative prognosis typically refers to a negative clinical outcome or outlook, such as aggressive disease, disease progression and hospitalisation.

Suitably, the method of the present aspect further includes the step of diagnosing said subject as having a less favourable prognosis or a more favourable prognosis. In one embodiment, a relative or absolute increase in the expression of the target nucleic acid is diagnostic of a less favourable or poor prognosis in the subject. In a further embodiment, a relative or absolute decrease in the expression of the target nucleic acid is diagnostic of a more favourable prognosis in the subject. It will also be understood that a target nucleic acid expression level may be used to identify those poorer prognosis patients, such as those with more aggressive disease, who may benefit from one or more additional anti-viral and/or anti-inflammatory therapeutic agents to the typical or standard SARS-CoV-2 treatment regime for that particular patient group.

Suitably, the biological sample is that hereinbefore described, such as cells, blood, serum, plasma, saliva, cerebrospinal fluid, urine, stool, sputum, nasopharyngeal aspirates or swabs obtained from the subject.

For the present method, the portion of subgenomic mRNA suitably comprises at least a portion of a leader sequence, such as that hereinbefore described.

In various embodiments, the portion of subgenomic mRNA comprises at least a portion of an E gene or gene product thereof, such as that provided herein.

In some embodiments, the portion of subgenomic mRNA of SARS-CoV-2 comprises, consists of or consists essentially of a nucleotide sequence set forth in SEQ ID NO:6 or a fragment or variant thereof.

Suitably, the step of amplifying the nucleic acid includes using one or more primers that hybridize or anneal to (e.g., are complementary or substantially complementary to) the portion of SARS-CoV-2 subgenomic mRNA or a cDNA sequence derived therefrom. In some embodiments, amplifying the target nucleic acid includes using a forward primer that hybridizes to the portion of the leader sequence or a cDNA sequence derived therefrom. In other embodiments, amplifying the target nucleic acid includes using a reverse primer that hybridizes to the portion of the E gene or a cDNA sequence derived therefrom. In specific embodiments, amplifying the target nucleic acid includes using a forward primer comprising, consisting of or consisting essentially of a nucleotide sequence set forth in SEQ ID NO:4, a nucleotide sequence complementary thereto or a fragment or variant thereof and/or a reverse primer comprising, consisting of or consisting essentially of a nucleotide sequence set forth in SEQ ID NO:5, a nucleotide sequence complementary thereto or a fragment or variant thereof.

Suitably, amplifying the target nucleic acid utilizes reverse transcriptase-polymerase chain reaction (RT-PCR). In this regard, the methods of the invention may further include the steps of obtaining RNA from the sample, the biological sample or one or more cells and reverse transcribing the RNA to obtain complementary DNA (cDNA).

It is envisaged that the step of detecting the presence or absence of the nucleic acid amplified in step (a) can be performed by any means or method known in the art, inclusive of those hereinbefore provided. In particular embodiments, the presence or absence of the nucleic acid is detected at least in part using high resolution melt (HRM) analysis.

In various embodiments, the present method includes the further step of detecting the presence or absence of SARS-CoV-2 genomic RNA in the sample, such as by any method known in the art inclusive of those described herein.

Suitably, detecting the presence of the target nucleic acid indicates replication of SARS-CoV-2 in the biological sample from the subject. It is further envisaged that the step of detecting the presence or absence of subgenomic mRNA of SARS-CoV-2 could include determining an expression level thereof. Such an expression level may subsequently be used to determine a level of replication of the SARS-CoV-2 virus in the sample, such as hereinafter described.

Accordingly, the method of present aspect further includes the step of determining a level of replication of SARS-CoV-2 in the biological sample from the subject. This may be achieved at least in part by measuring an expression level of the target nucleic acid amplified in step (a), wherein the expression level of the target nucleic acid indicates or correlates with the level of replication of SARS-CoV-2 in the biological sample.

The terms “determining”, “measuring”, “evaluating”, “assessing” and “assaying” are used interchangeably herein and may include any form of measurement known in the art, such as those described hereinafter.

Determining, assessing, evaluating, assaying or measuring the expression level the target nucleic acid, such as RNA, mRNA and cDNA, may be performed by any technique known in the art. These may be techniques that include nucleic acid sequence amplification, nucleic acid hybridization, nucleotide sequencing, mass spectroscopy and combinations of any these.

Nucleic acid amplification techniques typically include repeated cycles of annealing one or more primers to a “template” nucleotide sequence under appropriate conditions and using a polymerase to synthesize a nucleotide sequence complementary to the target, thereby “amplifying” the target nucleotide sequence. Nucleic acid amplification techniques are well known to the skilled addressee, and include but are not limited to polymerase chain reaction (PCR); strand displacement amplification (SDA); rolling circle replication (RCR); nucleic acid sequence-based amplification (NASBA), Q-β replicase amplification; helicase-dependent amplification (HAD); loop-mediated isothermal amplification (LAMP); nicking enzyme amplification reaction (NEAR) and recombinase polymerase amplification (RPA), although without limitation thereto. As generally used herein, an “amplification product” refers to a nucleic acid product generated by a nucleic acid amplification technique.

PCR includes quantitative and semi-quantitative PCR, real-time PCR, allele-specific PCR, methylation-specific PCR, asymmetric PCR, nested PCR, multiplex PCR, touch-down PCR, digital PCR and other variations and modifications to “basic” PCR amplification.

Nucleic acid amplification techniques may be performed using DNA or RNA extracted, isolated or otherwise obtained from a sample (e.g., a biological sample), a cell or tissue source. In other embodiments, nucleic acid amplification may be performed directly on appropriately treated cell or tissue samples.

Nucleic acid hybridization typically includes hybridizing a nucleotide sequence, typically in the form of a probe, to a target nucleotide sequence under appropriate conditions, whereby the hybridized probe-target nucleotide sequence is subsequently detected. Non-limiting examples include Northern blotting, slot-blotting, in situ hybridization and fluorescence resonance energy transfer (FRET) detection, although without limitation thereto. Nucleic acid hybridization may be performed using DNA or RNA extracted, isolated, amplified or otherwise obtained from a cell or tissue source or directly on appropriately treated cell or tissue samples.

It will also be appreciated that a combination of nucleic acid amplification and nucleic acid hybridization may be utilized.

As will be appreciated by the skilled person, the level of replication may indicate or correlate with a more or less favourable prognosis for the SARS-CoV-2 infection in the subject (e.g., indicate whether the subject has an ongoing infection with viral replication or is no longer infected or infectious with SARS-CoV-2). By way of example, a high or increased level of replication can indicate or correlate with a less favourable prognosis for the SARS-CoV-2 infection and a low or decreased level of replication can indicate or correlate with a more favourable prognosis for the SARS-CoV-2 infection.

As will be understood by a skilled person, the expression level of the target nucleic acid may be relatively (i) higher, increased or greater; or (ii) lower, decreased or reduced when compared to an expression level in a control or reference sample, or to a threshold expression level. In one embodiment, an expression level may be classified as high, increased or greater if it exceeds a mean and/or median expression level of a reference population. In one embodiment an expression level may be classified as low, decreased or reduced if it is less than the mean and/or median expression level of the reference population. In this regard, a reference population may be a group of subjects who are infected with SARS-CoV-2 and are at the same stage of infection as said subject for which the expression level is determined.

Terms such as “high”, “increased” and “greater” as used herein refer to an elevated amount or level of the target nucleic acid, such as in a biological sample, when compared to a control or reference level or amount. The expression level of the target nucleic acid may be relative or absolute. In some embodiments, the expression of the target nucleic acid is higher, increased or greater if its level of expression is more than about 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400% or at least about 500% above the level of expression of a target nucleic acid in a control, reference or threshold level or amount.

The terms, “low”, “reduced” and “decreased”, as used herein refer to a lower amount or level of the target nucleic acid, such as in a biological sample, when compared to a control or reference level or amount. The expression level of the target nucleic acid may be relative or absolute. In some embodiments, the expression of the target nucleic acid is lower, reduced or decreased if its level of expression is less than about 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the level or amount of expression of the target nucleic acid in a control, reference or threshold level or amount.

The term “control sample” may refer to a biological sample from a (healthy) non-diseased individual not having SARS-CoV-2 or from the same subject at one or more time points, such as prior to SARS-CoV-2 infection. In one embodiment, the control sample may be from a subject known to be free of SARS-CoV-2. Alternatively, the control sample may be from a subject previously infected with SARS-CoV-2. The control sample may be a pooled, average or an individual sample. An internal control is a marker from the same biological sample being tested.

As used herein, an expression level may be an absolute or relative amount of an expressed nucleic acid. Accordingly, in some embodiments, the expression level of the target nucleic is compared to a control level of expression, such as the level of expression of one or a plurality of “housekeeping” genes or nucleic acids in the biological sample of the subject.

In further embodiments, the expression level of the target nucleic acid is compared to a threshold level of expression. A threshold level of expression is generally a quantified level of expression of the target nucleic acid. Typically, an expression level of the target nucleic acid in a sample that exceeds or falls below the threshold level of expression is predictive of a particular disease state or outcome, such as clearance of the SARS-CoV-2 virus. The nature and numerical value (if any) of the threshold level of expression will typically vary based on the method chosen to determine the expression of the target nucleic thereof, used in determining, for example, a prognosis in the subject.

A person of skill in the art would be capable of determining the threshold level of the target nucleic acid expression in a sample that may be used in determining, for example, a prognosis, using any method of measuring gene expression known in the art, such as those described herein. In one embodiment, the threshold level is a mean and/or median expression level (median or absolute) of the target nucleic acid in a reference population, that, for example, have the same SARS-CoV-2 serotype and stage of the infection as said subject for which the expression level is determined. Additionally, the concept of a threshold level of expression should not be limited to a single value or result. In this regard, a threshold level of expression may encompass multiple threshold expression levels that could signify, for example, a high, medium, or low probability of, for example, disease progression and/or hospitalisation, as described herein.

In another aspect, the invention relates to a method for identifying an agent capable of altering or modulating replication of SARS-CoV-2 in one or more cells infected therewith, said method including the steps of:
(a) amplifying a target nucleic acid from a portion of subgenomic mRNA of SARS-CoV-2 from the one or more cells treated with the agent; and
(b) measuring an expression level of the target nucleic acid amplified in step (a), wherein the expression level of the target nucleic acid indicates or correlates with a level of replication of SARS-CoV-2 in the one or more cells.

As would be understood by the skilled person, the expression level of the target nucleic acid and/or the level of replication in the one or more cells is deemed to be “altered” or “modulated” when the amount or level thereof is increased or up regulated or decreased or down regulated, as defined herein. For example, “altering or modulating replication” of a virus includes increasing and/or decreasing the quantity of any one or more of the steps of adsorption (e.g., receptor binding) to a cell, entry into a cell (such as by endocytosis), introducing the viral genome sequence into the cell, uncoating the viral genome, initiating transcription of genomic RNA, producing subgenomic mRNA, directing expression of SARS-CoV-2 encapsidation proteins, encapsidating the replicated viral nucleic acid sequence with the encapsidation proteins into a viral particle, release of the encapsidated virus from the cell, and infection of other cells by the released virus.

Suitably, the present method further includes the step of determining the level of replication in the one or more cells treated with the agent at least in part by the expression level of the target nucleic acid measured in step (b).

Suitably, steps (a) and (b) of the present method may be performed by any means known in the art, such as those hereinbefore described. In particular embodiments, the step of measuring the expression level of the target nucleic acid may include detecting the presence or absence of the target nucleic acid.

The one or more cells (or tissues, organs, organoids or the like derived therefrom) suitably support replication of SARS-CoV-2 in the absence of any substantial adverse or cytopathic effects. In particular embodiments, the one or more cells may comprise mammalian cells (e.g., human cells), insect cells, avian cells and/or plant cells. The present invention further contemplates the use of transgenic cells (i.e., cells that has been transformed to contain a transgene). As will be appreciated, coronaviruses can grow in a variety of transformed and primary mammalian cell lines, such as HEK-293T, Huh-7, MvlLu, pRHMK and pCMK. A primary determinant of infection is the expression of specific host proteins that function as receptors to bind the coronavirus S protein and initiate steps in virus entry and uncoating of genome RNA for replication in the host-cell cytoplasm. However, methods to either express receptor in naturally non-permissive cells, mechanisms to bypass the receptor requirement, and virus adaptation have allowed coronaviruses to grow in a very wide variety of cell types of divergent species. Coronaviruses generally grow well in culture under standard media, pH, temperature and supplement conditions.

Suitably, the expression level of the target nucleic acid and/or the level of replication of SARS-CoV-2 in the one or more cells treated with the agent may be compared to one or more control, reference or threshold levels thereof, such as those hereinbefore described. In some embodiments, the expression level of the target nucleic acid and/or the level of replication of the cells treated with the agent is compared to that for corresponding cells that have not been treated with the agent. In some embodiments, detecting or determining a decrease or reduction in the expression level of the target nucleic acid and/or the level of replication in the one or more cells treated with the agent compared to, for example, said cells not treated with the agent identifies the agent as being efficacious in reducing replication of SARS-CoV-2 in the one or more cells. Alternatively, detecting or determining no change or an increase or upregulation in the expression level of the target nucleic acid and/or the level of replication in the one or more cells treated with the agent compared to, for example, said cells not treated with the agent identifies the agent as not being efficacious in reducing replication of SARS-CoV-2 in the one or more cells.

By way of example, the present method may include determining the expression level of the target nucleic acid and/or the level of replication of: (i) a first population of the one or more cells treated with the agent; and (ii) a second population of the one or more cells not treated with the agent; and comparing the expression level of the target nucleic acid and/or the level of replication of the first and second population of cells. In this regard, a change or difference between the level of replication determined for the first and second populations of the cells indicates that the agent is capable of altering replication of SARS-CoV-2 (e.g., a decrease or reduction in the expression level of the target nucleic acid and/or the level of replication in the first population of the one or more cells treated with the agent compared to the second population of cells identifies the agent as being efficacious in reducing replication of SARS-coronavirus in the one or more cells).

In alternative embodiments, the present method comprises determining the expression level of the target nucleic acid and/or the level of replication of the cells at respective first and second time points; and comparing the expression level of the target nucleic acid and/or the level of replication of the cells at the first and second time points, wherein a change between the level of replication measured at the first and second time points indicates that the agent is capable of altering replication of SARS-CoV-2. In this regard, a change or difference between the level of replication determined for the first and second time points indicates that the agent is capable of altering replication of SARS-CoV-2 (e.g., a decrease or reduction in the expression level of the target nucleic acid and/or the level of replication in the one or more cells treated with the agent from the first time point to the second time point identifies or indicates the agent as being efficacious in reducing replication of SARS-coronavirus in the one or more cells).

Suitably, the current method further includes the initial steps of: infecting the one or more cells with SARS-CoV-2 and/or treating the one or more cells with the agent.

The term “agent” refers to any type of molecule (for example, a peptide, nucleic acid, carbohydrate, lipid, organic, and inorganic molecule, etc.) obtained from any source (for example, plant, animal, and environmental source, etc.), or prepared by any method (for example, purification of naturally occurring molecules, chemical synthesis, and genetic engineering methods, etc.). In some embodiments, the agent may be referred to as a “test compound” or “test agent”, which can refer to any chemical entity, pharmaceutical, drug, and the like that can be used to treat or prevent a disease, illness, sickness, or disorder of bodily function. Agents comprise both known and potential therapeutic compounds. An agent can be determined to be therapeutic by screening using the present method. A “known therapeutic compound” refers to a therapeutic compound that has been shown (e.g., through animal trials or prior experience with administration to humans) to be effective in such treatment or prevention. In other words, a known therapeutic compound is not limited to a compound efficacious in the treatment of SARS-CoV-2 infection. Agents are exemplified by, but not limited to, vaccines, antibodies, nucleic acid sequences such as ribozyme sequences, and other agents as further described herein. In one embodiment, the agent is an antibody or a small molecule that is specific for one or more SARS-CoV-2 antigens.

In certain embodiments, the portion of subgenomic mRNA suitably comprises at least a portion of a leader sequence, such as that previously disclosed herein.

In some embodiments, the portion of subgenomic mRNA suitably comprises at least a portion of an E gene or gene product thereof, such as that hereinbefore provided.

In various embodiments, the portion of subgenomic mRNA of SARS-CoV-2 comprises, consists of or consists essentially of a nucleotide sequence set forth in SEQ ID NO:6 or a fragment or variant thereof.

Suitably, the step of amplifying the nucleic acid includes using one or more primers that hybridize or anneal to (e.g., are complementary or substantially complementary to) the portion of SARS-CoV-2 subgenomic mRNA or a cDNA sequence derived therefrom. In some embodiments, amplifying the target nucleic acid includes using a forward primer that hybridizes to the portion of the leader sequence or a cDNA sequence derived therefrom. In other embodiments, amplifying the target nucleic acid includes using a reverse primer that hybridizes to the portion of the E gene or a cDNA sequence derived therefrom. In specific embodiments, amplifying the target nucleic acid includes using a forward primer comprising, consisting of or consisting essentially of a nucleotide sequence set forth in SEQ ID NO:4, a nucleotide sequence complementary thereto or a fragment or variant thereof and/or a reverse primer comprising, consisting of or consisting essentially of a nucleotide sequence set forth in SEQ ID NO:5, a nucleotide sequence complementary thereto or a fragment or variant thereof.

Suitably, in respect of the above aspects, amplifying the target nucleic acid utilizes reverse transcriptase-polymerase chain reaction (RT-PCR). In this regard, the methods of the invention may further include the steps of obtaining RNA from the sample, the biological sample or one or more cells and reverse transcribing the RNA to obtain complementary DNA (cDNA).

In some embodiments, the methods described generally herein are performed, at least in part, by a processing system, such as a suitably programmed computer system. A stand-alone computer, with the microprocessor executing applications software allowing the above-described methods to be performed, may be used. Alternatively, the methods can be performed, at least in part, by one or more processing systems operating as part of a distributed architecture. For example, a processing system can be used to detect the presence of the target nucleic acid by detecting the hybridization of a probe thereto or the presence of a suitable HRM curve. A processing system also can be used to determine whether a sample (or a subject from which the sample is obtained) is positive or negative for replication of SARS-CoV-2 (e.g., a subject is infectious) on the basis of detection of the target nucleic acid. In some examples, commands inputted to the processing system by a user may assist the processing system in making these determinations.

In specific embodiments, a processing system includes at least one microprocessor, a memory, an input/output device, such as a keyboard and/or display, and an external interface, interconnected via a bus. The external interface can be utilised for connecting the processing system to peripheral devices, such as a communications network, database, or storage devices. The microprocessor can execute instructions in the form of applications software stored in the memory to allow the target nucleic acid detection and/or virus replication detection process to be performed, as well as to perform any other required processes, such as communicating with the computer systems. The application software may include one or more software modules, and may be executed in a suitable execution environment, such as an operating system environment, or the like.

In yet another aspect, the invention relates to an isolated probe, tool or reagent capable of detecting SARS-CoV-2 in a sample, wherein the probe, tool or reagent is capable of binding, detecting or identifying the presence or absence of subgenomic mRNA of SARS-CoV-2.

The probe, tool or reagent may be, but is not limited to, an oligonucleotide, a primer, a nucleic acid, a polynucleotide, DNA, cDNA, RNA, a peptide or a polypeptide. These may be, for example, single stranded or double stranded, naturally occurring, isolated, purified, chemically modified, recombinant or synthetic.

The probe, tool or reagent may be detectably labelled. A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labelled, so as to incorporate the label into the amplification product.

In certain embodiments, the at least one probe, tool or reagent is for specifically binding, detecting or identifying at least a portion of subgenomic mRNA of SARS-CoV-2 in a sample as provided herein (e.g., the portion of subgenomic mRNA set forth in SEQ ID NO:6).

One of skill in the art could readily design, produce or manufacture a wide range of gene/allele-based and gene product-based probes, tools, reagents, methods and assays based on the information provided herein and especially in FIGS. 1A-1F and 2.

Generally speaking, such probes, tools or reagents based on or developed in view of the SNPs outlined in the present specification may, for example, specifically bind, detect, identify, characterise or quantify the gene or part of the gene, or other gene products or parts thereof.

Generally speaking, such probe, tool or reagent can be for detection of a polymorphism for example at the genomic level, or at the transcription level.

Generally speaking, such probe, tool or reagent can also be an antibody or other type of molecule or chemical entity capable of detecting the gene or gene product (such as RNA).

More specifically, probes, tools and reagents may include, but are not limited to, the following:
1. An isolated, purified, synthetic or recombinant form of a portion of subgenomic mRNA of SARS-CoV-2, or a fragment thereof, including a fragment containing a SNP or sequence of interest—single stranded or double stranded.
2. A non-naturally occurring polynucleotide, recombinant polynucleotide, oligonucleotide or cDNA form of subgenomic mRNA of SARS-CoV-2, or a fragment thereof, including a fragment containing a SNP or sequence of interest—single stranded or double stranded.
3. An expression vector, recombinant cell or biological sample comprising the nucleic acid or polynucleotide of 1 or 2 above.

The probe, tool or reagent can be, but is not limited to, an antibody or other type of molecule or chemical entity capable of detecting the gene or gene product (RNA or polypeptide).

The at least one probe, tool or reagent can be any number or combination of the above, and the number and combination will depend on the desired result to be achieved—e.g. detection of a polymorphism at the genomic level (genotyping) or at the RNA level.

Suitably, the probe, tool or reagent is or comprises an oligonucleotide or primer comprising, consisting of or consisting essentially of a nucleic acid sequence as set forth in at least one of SEQ ID NOs: 4 and 5, a nucleic acid sequence complementary thereto or a fragment or variant thereof. In one embodiment, the isolated probe, tool or reagent comprises or consists of a nucleotide sequence having at least 90%, at least 91%, at least 92%, at least 93%, at least 94%, at least 95%, at least 96%, at least 97%, at least 98%, at least 99% or 100% sequence homology or identity with the sequence as set forth in at least one of SEQ ID NOs: 4 and 5.

In yet another aspect, the invention provides an isolated oligonucleotide or primer comprising, consisting of or consisting essentially of a nucleic acid sequence as set forth in any one of SEQ ID NOs:4 and 5, a nucleotide sequence complementary thereto or a fragment or variant thereof.

The term “oligonucleotide” as used herein refers to a polymer composed of a multiplicity of nucleotide residues (deoxynucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length generally from about 10 to 35 nucleotide residues (e.g., about 10, 11, 12, 13, 14, 15, 16, 17, 18, 19, 20, 21, 22, 23, 24, 25, 26, 27, 28, 29, 30, 31, 32, 33, 34 and 35 nucleotides in length including any range therein), but the term can refer to molecules of any length, although the term “polynucleotide” or “nucleic acid” is typically used for large oligonucleotides.

In one embodiment, the isolated oligonucleotide comprises or consists of a nucleotide sequence having at least 90%, at least 91%, at least 92%, at least 93%, at least 94%, at least 95%, at least 96%, at least 97%, at least 98%, at least 99% or 100% sequence homology or identity with the sequence as set forth in at least one of SEQ ID NOs: 4 and 5.

In a related aspect, the invention resides in an array comprising the isolated oligonucleotide hereinbefore described.

In a further related aspect, the invention relates to a biochip comprising a solid substrate and the isolated oligonucleotide described herein.

In this regard, the present invention contemplates the use of an array of oligonucleotides, wherein discrete positions on the array are complementary to one or more of the sequences of the present invention or described herein, e.g. oligonucleotides of at least 12 nt, at least about 15 nt, at least about 18 nt, at least about 20 nt, or at least about 25 nt, or longer, and including the sequence flanking the polymorphic position. Such an array may comprise a series of oligonucleotides, each of which can specifically hybridize to a different portion of genomic and/or subgenomic mRNA of SARS-CoV-2 and optionally additional viruses, such as other coronavirus strains or isolates. For examples of arrays, see Hacia et al., 1996, Nat. Genet. 14: 441-447 and De Risi et al, 1996, Nat. Genet. 14: 457-460.

A number of methods are available for creating micro-arrays of biological samples, such as arrays of DNA samples to be used in DNA hybridization assays. Examples of such arrays are discussed in detail in PCT Application No. WO95/35505; U.S. Pat. No. 5,445,934; and Drmanac et al., 1993, Science 260: 1649-1652. Yershov et al., 1996, Genetics 93: 4913-4918 describes an alternative construction of an oligonucleotide array. The construction and use of oligonucleotide arrays are reviewed by Ramsay (Ramsay, 1998, Nature Biotech. 16: 40-44).

Methods of using high-density oligonucleotide arrays for identifying polymorphisms within nucleotide sequences are known in the art. For example, Milosavljevic et al., 1996 describe DNA sequence recognition by hybridization to short oligomers (see also, Drmanac et al., 1998, Nature Biotech. 16: 54-58 and Drmanac and Drmanac, 1999, Methods Enzymol. 303: 165-178). The use of arrays for identification of unknown mutations is proposed by Ginot (Ginot, 1997, Human Mutation 10:1-10).

Detection of known mutations is described in Hacia et al., 1996, Nat. Genet. 14: 441-447; Cronin, et al., 1996, Human Mut. 7: 244-255; and others. The use of arrays in genetic mapping is discussed in Chee, et al., 1996, Science 274: 610-613; Sapolsky and Lishutz, 1996, Genomics 33: 445-456; and Shoemaker et al., 1996, Nat. Genet. 14: 450-456.

Quantitative monitoring of gene expression patterns with a complementary DNA microarray is described in Schena et al., 1995, Science 270: 467; and DeRisi et al., 1997, Science 270:680-686. Wodicka et al., 1997 (Wodicka et al, 1997, Nat. Biotech. 15: 1-15) performs genome wide expression monitoring in S. cerevisiae.

High-density microarrays of oligonucleotides are known in the art and are commercially available. The sequence of oligonucleotides on the array will correspond to a known target sequence.

The length of oligonucleotide present on the array is an important factor in how sensitive hybridization will be to the presence of a mismatch. Usually oligonucleotides will be at least about 12 nt in length, more usually at least about 15 nt in length, preferably at least about 20 nt in length and more preferably at least about 25 nt in length, and will be not longer than about 35 nt in length, usually not more than about 30 nt in length.

Methods of producing large arrays of oligonucleotides are described in U.S. Pat. Nos. 5,134,854 and 5,445,934 using light-directed synthesis techniques. Using a computer-controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in International Publication WO 95/35505.

Microarrays can be scanned to detect hybridization of the labelled genome samples. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that may be used in the subject methods is described in U.S. Pat. No. 5,631,734. A scanning laser microscope is described in Shalon, et al., 1996, Genome Res. 6: 639. A scan, using the appropriate excitation line, is performed for each fluorophore used. The digital images generated from the scan are then combined for subsequent analysis. For any particular array element, the ratio of the fluorescent signal from one nucleic acid sample is compared to the fluorescent signal from the other nucleic acid sample, and the relative signal intensity determined.

Methods for analysing the data collected by fluorescence detection are known in the art. Data analysis includes the steps of determining fluorescent intensity as a function of substrate position from the data collected, removing outliers, i.e., data deviating from a predetermined statistical distribution, and calculating the relative binding affinity of the targets from the remaining data. The resulting data may be displayed as an image with the intensity in each region varying according to the binding affinity between targets and probes.

Nucleic acid analysis via microchip technology is also applicable to the present invention. In this technique, thousands of distinct oligonucleotide probes can be applied in an array on a silicon chip. A nucleic acid to be analysed is fluorescently labelled and hybridized to the probes on the chip. It is also possible to study nucleic acid-protein interactions using these nucleic acid microchips. Using this technique, one can determine the presence of mutations, sequence the nucleic acid being analysed, or measure expression levels of a gene of interest. The method is one of parallel processing of many, even thousands, of probes at once and can tremendously increase the rate of analysis.

An array-based tiling strategy useful for detecting SNPs is described in EP 785280. Briefly, arrays may generally be “tiled” for a large number of specific polymorphisms. “Tiling” refers to the synthesis of a defined set of oligonucleotide probes that are made up of a sequence complementary to the target sequence of interest, as well as preselected variations of that sequence, e.g., substitution of one or more given positions with one or more members of the basis set of monomers, i.e., nucleotides. Tiling strategies are further described in PCT application No. WO 95/11995. In some embodiments, arrays are tiled for a number of specific SNPs. In particular, the array is tiled to include a number of detection blocks, each detection block being specific for a specific SNP or a set of SNPs. For example, a detection block may be tiled to include a number of probes that span the sequence segment that includes a specific SNP. To ensure probes that are complementary to each allele, the probes are synthesized in pairs differing at the SNP position. In addition to the probes differing at the SNP position, monosubstituted probes are also generally tiled within the detection block. Such methods can readily be applied to the SNP information disclosed herein.

These monosubstituted probes have bases at and up to a certain number of bases in either direction from the polymorphism, substituted with the remaining nucleotides (selected from A, T, G, C and U). Typically, the probes in a tiled detection block will include substitutions of the sequence positions up to and including those that are 5 bases away from the SNP. The monosubstituted probes provide internal controls for the tiled array, to distinguish actual hybridization from artificial cross-hybridization. Upon completion of hybridization with the target sequence and washing of the array, the array is scanned to determine the position on the array to which the target sequence hybridizes. The hybridization data from the scanned array is then analysed to identify which allele or alleles of the SNP are present in the sample. Hybridization and scanning may be carried out as described in PCT application No. WO 92/10092 and WO 95/11995 and U.S. Pat. No. 5,424,186.

Thus, in some embodiments, the chips may comprise an array of nucleic acid sequences of fragments of about 15 nucleotides in length and the sequences complementary thereto, or a fragment thereof, the fragment comprising at least about 8 consecutive nucleotides, preferably 10, 15, 20, more preferably 25, 30, 40, 47, or 50 consecutive nucleotides and containing a polymorphic base, such as those described herein. In some embodiments the polymorphic base is within 5, 4, 3, 2, or 1 nucleotides from the centre of the polynucleotide, more preferably at the centre of the polynucleotide. In other embodiments, the chip may comprise an array containing any number of polynucleotides of the present invention.

An oligonucleotide may be synthesized on the surface of the substrate by using a chemical coupling procedure and an ink jet application apparatus, as described in PCT application WO95/251116. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536, 6144 or more oligonucleotides, or any other number which lends itself to the efficient use of commercially available instrumentation.

Using such arrays, the present invention provides methods of detecting replication of SARS-CoV-2 in a sample. Such methods comprise incubating a test sample with an array comprising one or more oligonucleotide probes corresponding to a portion of subgenomic mRNA (and optionally genomic RNA) of SARS-CoV-2, such as those provided for the present invention, and assaying for binding of a nucleic acid from the test sample with one or more of the oligonucleotide probes. Such assays will typically involve arrays comprising oligonucleotide probes corresponding to multiple positions and/or allelic variants of the subgenomic mRNA.

Conditions for incubating a nucleic acid molecule with a test sample vary. Incubation conditions depend on the format employed in the assay, the detection methods employed, and the type and nature of the nucleic acid molecule used in the assay. One skilled in the art will recognize that any one of the commonly available hybridization, amplification or array assay formats can readily be adapted to employ the methods disclosed herein.

In a final aspect, the invention provides a kit or assay for detecting replication of SARS-CoV-2 in a sample, said kit or assay comprising: the isolated oligonucleotide described herein; the array described herein; and/or the biochip described herein.

In this regard, all the essential materials and reagents required for performing the methods described herein, such as amplifying and/or detecting a portion of subgenomic mRNA (and optionally genomic RNA) of SARS-CoV-2, may be assembled together in a kit. The kits may also optionally include appropriate reagents for detection of labels, positive and negative controls, fluorescent dyes (e.g., for HRM analysis), washing solutions, blotting membranes, microtitre plates, dilution buffers and the like. For example, a nucleic acid-based detection kit for the identification of polymorphisms may include one or more of the following: (i) nucleic acid (e.g., RNA or cDNA) from a SARS-CoV-2 strain or isolate (which may be used as a positive control); and (ii) a primer and/or probe that specifically hybridizes to at least aportion of subgenomic mRNA of SARS-CoV-2 to be analysed, and optionally one or more other markers. Also included may be enzymes suitable for amplifying nucleic acids, such as the target nucleic acid, including various polymerases (Reverse Transcriptase, Taq, Sequenase™ DNA ligase etc. depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe. The kit can also feature various devices and reagents for performing one of the assays described herein; and/or printed instructions for using the kit to identify the presence of or expression level of the target nucleic acid, and hence replication of the SARS-CoV-2 virus, as defined herein.

In particular embodiments, the kit includes a pair of oligonucleotides (e.g., comprising a forward/sense primer and an antisense/reverse primer), wherein at least one of the pair of oligonucleotides comprises, consists of or consists essentially of the nucleic acid sequence as set forth in any one of SEQ ID NOs: 4 and 5, a nucleic acid sequence complementary thereto or a fragment or variant thereof. In some embodiments, the kit further comprises a second pair of primers for detecting the presence or absence of SARS-CoV-2 genomic RNA. (e.g., the second pair of primers anneal or hybridize to a cDNA sequence derived or obtained from SARS-CoV-2 genomic RNA in a sample, a biological sample or one or more cells by RT-PCR).

In particular embodiments, the kit includes instructions for use, such as instructions for using the pair of oligonucleotides in RT-PCR and high resolution melt analysis.

Suitably, the kit is for use in one or more of the methods described herein.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

The disclosure of every patent, patent application, accession number and publication cited herein is hereby incorporated herein by reference in its entirety.

In order that the invention may be readily understood and put into practical effect, particular preferred embodiments will now be described by way of the following non-limiting examples.

### EXAMPLES

### Example 1

The present Example illustrates an embodiment of a one-step PCR test that can be utilized to specifically detect the replication of SARS-CoV-2 in samples obtained from patients.

### One-Step SARS-CoV-2 RT-PCR and High Resolution Melt Method

### RT-PCR Preparation

Table 1 shows the primers and reaction parameters used in the assay. Table 2 illustrates a reaction setup for a one-step Reverse-Transcriptase Real-time PCR run profile, showing the cycling parameters utilised to identify replicating SARS-CoV-2. The following occurs within a positive pressure environment.
1. Thaw, vortex, and centrifuge the reaction components.
2. Add all the reaction components except the viral RNA sample to a tube as per the reaction setup table.
3. Multiply to accommodate the number of reactions required.
4. Vortex and centrifuge the mixture volume.
5. For a single reaction, add 19 μL of mixture to the real-time PCR tube.
6. Vortex and centrifuge the RNA template.
7. Add 6 μL of the RNA template to the PCR tube per reaction.
8. Apply the lids to the real-time PCR tubes and ensure that they are sealed.

### RT-PCR Machine Operation

1. Load the reaction tubes and fill the remaining slots with balance tube (containing water).
2. In the Samples table fill in:
   a. The Name column with sample identities.
   b. The Type column with the sample type (i.e. Standard, Control, NTC).
   c. The Assay column will autofill when selecting the sample type to match your assigned assay.
   d. If required, the Standards Concentration column is where you will input your concentrations. Unit of measurement can be selected at the top of the column
3. Once sample details have been provided, the PCR is ready for operation, for example, using the run profile conditions provided in FIG. 6 for the MIC platform.
   Interpretation ofART-PCR results
1. Utilise the relevant Real-Time PCR machine's application for High-Resolution Melt curve analysis.
2. Utilise the relevant Real-Time PCR machine's application to determine the limit of detection of the assay based on a standard curve generated from serially diluted known concentration ofnreference gene target.

### Results

### Interpreting Test Results

Exemplary results for the above PCR test for patients determined to be either negative or positive for SARS-CoV-2 and either positive or negative for virus replication are shown in FIG. 5.

TABLE 1
Primers and reaction parameters

| InfectID-CoV-2 assays | | | | | Amplicon | | Reaction |
|---|---|---|---|---|---|---|---|
| Primer | | | | | | Amplicon TM | Optimal |
| | | Primer | Primer | Target | Amplicon- | (nearest | annealing |
| Primer | Seq 5′- | length | Tm | | length | neighbour) | temp |
| IC_ | CTTGGCTATTCAGTTGTT | 24 bp | 54.6° C. | RPP30 | 177 bp | 77.8° C. | 58.2° C. |
| RPP30-F | GCTATC (SEQ ID NO: 2) | | | | | | |
| IC | GTGAGATGGATCCGAGA | 24 bp | 53.8° C. | RPP30 | | | |
| RPP30-R | CAATAAT (SEQ ID NO: 3) | | | | | | |
| sgRNA- | CGATCTCTTGTAGATCTG | 23 bp | 53.3° C. | sgF- | 171 bp | 80.8° C. | 57.7º C. |
| F | TTCTC (SEQ ID NO: 4) | | | gene | | | |
| sgRNA- | ATATTGCAGCAGTACGC | 22 bp | 56.1° C. | sgF- | | | |
| R | ACACA (SEQ ID NO: 5) | | | gene | | | |

TABLE 2
Reaction Setup

| Components | Volume (μL) |
|---|---|
| Water | 0.75 |
| Source Template | 6 |
| Accumelt_sg Forward Primer | 2 |
| Accumelt_sg Reverse Primer | 2 |
| Accumelt (2X) | 12.5 |
| qScript XLT 1-step RT (25x) | 1 |
| MgCl2 | 0.75 |
| Total Volume (μL) | 25 |

### Example 2

The present sample illustrates an embodiment of a Quantitative Real-time PCR (gRT-PCR) method for the detection of active SARS-CoV-2 Subgenomic mRNAs (sg mRNA) assay using the method of Example 1 and utilising Mic qPCR hardware (Bio Molecular systems). The analysis software is Mic PCRv2.10.0.

Standard examples were made up with SARS-CoV-2 hCoV-19/Australia/VIC01/2020 (GeneBank accession number: MT007544.1). The virus had been passaged as follows: two passages in Vero cells-Kidney epithelial cells derived from African green monkey. A working stock was generated by four further passages in Vero E6 cells in virus growth media, which comprised Dulbecco's Modified Eagle's Medium-high glucose (DMEM; Thermo Fisher Scientific, Cat. No. 10313-021) supplemented with 1% (w/v) GlutaMAX (Thermo Fisher Scientific, Cat. No. 35050-061), 2% (v/v) FBS (Bovogen, Cat. No. SFBS) and 1% (v/v penicillin streptomycin (Thermo Fisher Scientific Cat. No. 15140-122).

The virus titre was determined using Tissue Culture Infective dose (TCID50/mL) assay. Briefly, 100 μL of virus was added to quadruplicate wells of a 96-well plate containing Vero E6 cells pre-seeded overnight at 1.5×10⁴ cells/well in virus growth media. The virus was titrated 1 in 3 across the plate. Plates were incubated for three days at 37° C. in a humidified 5% CO2 atmosphere, and virus-induced CPE scored visually. The TCID₅₀ of the virus was determined using the method of Reed-Muench (Reed, L. J., Muench, H. (1938). A simple method estimating fifty percent endpoints. The American Journal of Hygiene. 27:493-497). SARS-CoV-2 titred virus stock was heat inactivated at 65° C. for 30 minutes. The infectivity was then determined by TCID₅₀ assay.

### Limit of Detection

The lower limit of detection (LOD) of an individual analytical procedure describes the lowest quantity of analyte in a sample which can be qualitatively determined with suitable precision and accuracy. To determine this 30 PCR confirmed SARS-CoV-2 negative nasopharyngeal samples were obtained.

Samples were prepared using pooled clinical nasopharyngeal swab matrix from 20 individuals. The pooled matrix was previously SARS-CoV-2 PCR tested and confirmed to be negative. In the first part of the study, a total of six 10-fold dilutions of known concentrations of inactivated SARS-CoV-2 virus were prepared in the negative matrix and total viral RNA was extracted using the Maxwell RSC Viral Total Nucleic Acid Purification kit (Promega, Cat. No #AS1330) and the Maxwell nucleic extraction instrumentation (Promega) based on the manufacturers' instructions. Briefly, 330 μL of lysis buffer containing 30 μL Protease K was added to 300 μL of nasopharyngeal matrix. The sample was first incubated at room temperature for 10 minutes, followed by 10 minutes at 56° C. The sample was then added to the Maxwell cartridge ready for extraction. The Maxwell instrument is designed for use with pre-dispensed reagent cartridges and pre-programed purification procedures. The isolated RNA was then used for RT-PCR reaction, using a one-step real-time PCR procedure combined with High Resolution Melt curve analysis method according to the invention. Three extraction replicates were tested. The results are summarised in the following table:

The PCR reaction was set up as follows:

| | Vol | |
|---|---|---|
| Reagent | (μL) | |
| AC Mastermix (MicroBio) | 18 | μL |
| XLT Reverse transcriptase enzyme (25X) | 1 | μL |
| RNA Template | 6 | μL |
| Total Volume | 25 | μL |

Set up the thermocycler protocol was as follows:

| | | | | | | | Annealing and | | | | |
|---|---|---|---|---|---|---|---|---|---|---|---|
| CDNA synthesis | | | | Denaturation | | | extension | | | Melting curve | |
| 1 Cycle | | | | 40 Cycles | | | | | | 1 Cycle | |
| 48° | C. | 94° | C. | 94° | C. | 52 | C. | 72° | C. | 70° C. | 90° C. |

| | | | | | | | | | | |
|---|---|---|---|---|---|---|---|---|---|---|
| 20 | mins | 3 | mins | 2 | seconds | 2 | seconds | 5 | seconds | Increment 0.3° C./sec |

A positive result (detection of sg SARS-CoV-2 RNA) occurs when Mic PCR software automatically genotypes SARS-CoV-2 RNA sample based on Melting Temperature (Tm) values, using synthetic oligonucleotide as a positive control. A negative result occurs when the genotype cannot be determined by the Mic PCR software.

The LOD is defined as lowest concentration where at least 19 out of 20 replicates are positive.

TABLE 3
LOD study results from 10-fold dilution of
virus stock spiked into clinical matrix

| | | Detection | |
|---|---|---|---|
| | | Rate | |
| | Acceptance | (out of 3) | |
| TCID50/mL | Criteria | Sg mRNA | Result |
| 1.5 × 10⁵ | Valid Genotype | 3/3 | Positive |
| 1.5 × 10⁴ | based on Tm | 3/3 | Positive |
| 1.5 × 10³ # | | 3/3 | Positive |
| 1.5 × 102 | | 3/3 | Negative |
| 1.5 × 101 | | 3/3 | Negative |
| 1.5 | | 3/3 | Negative |
| NasoP only | | 0/3 | Negative |

# LOD 1.5 × 103 TCID50/mL

Data for the dilutions is as follows:

TABLE 4
Preliminary LOD study results from 10-fold dilution of virus stock

| | | | | Melting Temp | |
|---|---|---|---|---|---|
| TCID50/mL | Run | Ct | Genotype | (Tm) | Result |
| 1.5 × 10⁵ | 1 | 20.148 | SARS-CoV-2 | 80.75 | Positive |
| | 2 | 20.209 | SARS-CoV-2 | 80.6 | |
| | 3 | 20.806 | SARS-CoV-2 | 81.05 | |
| 1.5 × 10⁴ | 1 | 23.658 | SARS-CoV-2 | 80.84 | Positive |
| | 2 | 23.518 | SARS-CoV-2 | 80.44 | |
| | 3 | 24.101 | SARS-CoV-2 | 80.56 | |
| 1.5 × 10³ | 1 | 26.318 | SARS-CoV-2 | 80.39 | Positive |
| | 2 | 26.249 | SARS-CoV-2 | 80.54 | |
| | 3 | 26.371 | SARS-CoV-2 | 80.56 | |
| 1.5 × 10² | 1 | 27.148 | SARS-CoV-2 | 80.39 | Negative |
| | 2 | 28.520 | SARS-CoV-2 | 80.3 | |
| | 3 | 26.624 | Undetermined | 88.58 | |
| 1.5 × 10¹ | 1 | 29.062 | Undetermined | 77.85, 82.98, 87.01 | Negative |
| | 2 | 28.006 | Undetermined | 77.77, 87.32 | |
| | 3 | 27.955 | Undetermined | 77.92, 82.12 | |
| 1.5 | 1 | 28.126 | Undetermined | 77.83, 82.04, 88.56 | Negative |
| | 2 | 28.127 | Undetermined | 77.97, 87.20 | |
| | 3 | 26.630 | Undetermined | 82.20, 88.82 | |
| NasoP only | 1 | 27.172 | Undetermined | 78.05, 81.08 | Negative |
| | 2 | 27.008 | Undetermined | 78.00, 83.27, 87.24 | |
| | 3 | 27.860 | Undetermined | 78.02, 82.91, 88.55 | |

An additional twenty individual extraction replicates were tested, by spiking the pooled nasopharyngeal swab matrix with inactivated virus at the lowest tentative LOD (1.5×10³ TCID₅₀/mL). The results are summarised in the following Table.

TABLE 5
LOD study results from 10-fold dilution of
virus stock spiked into clinical matrix

| | | Detection | | |
|---|---|---|---|---|
| | | Rate | | |
| | | (out of 20) | | |
| TCID₅₀/mL | Acceptance Criteria | Sg mRNA | | Result |
| 1.5 × 10³ | Valid Genotype | 20/20 | (100%) | Positive |
| Negative | based on | 0/10 | (0%) | Negative |

| | |
|---|---|
| NasoP only | Tm |

Data for the replicates follows:

TABLE 6
LOD study results

| | | Melting | | |
|---|---|---|---|---|
| TCID50/mL | Run | temp (Tm) | Genotype | Result |
| 1 × LOD_rep 1 | 26.02 | 80.29 | SARS-CoV-2 | Positive |
| 1 × LOD_rep 2 | 26.06 | 80.32 | SARS-CoV-2 | Positive |
| 1 × LOD_rep 3 | 25.36 | 80.22 | SARS-CoV-2 | Positive |
| 1 × LOD_rep 4 | 27.03 | 80.2 | SARS-CoV-2 | Positive |
| 1 × LOD_rep 5 | 25.43 | 80.09 | SARS-CoV-2 | Positive |
| 1 × LOD_rep 6 | 26.24 | 80.14 | SARS-CoV-2 | Positive |
| 1 × LOD_rep 7 | 26.27 | 80.13 | SARS-CoV-2 | Positive |
| 1 × LOD_rep 8 | 26.14 | 80.11 | SARS-CoV-2 | Positive |
| 1 × LOD_rep 9 | 26.23 | 80.29 | SARS-CoV-2 | Positive |
| 1 × LOD_rep 10 | 26.41 | 80.33 | SARS-CoV-2 | Positive |
| 1 × LOD_rep 11 | 24.08 | 80.32 | SARS-CoV-2 | Positive |
| 1 × LOD_rep 12 | 24.70 | 80.45 | SARS-CoV-2 | Positive |
| 1 × LOD_rep 13 | 25.01 | 80.43 | SARS-CoV-2 | Positive |
| 1 × LOD_rep 14 | 25.06 | 80.52 | SARS-CoV-2 | Positive |
| 1 × LOD_rep 15 | 24.84 | 80.27 | SARS-CoV-2 | Positive |
| 1 × LOD_rep 16 | 24.33 | 80.18 | SARS-CoV-2 | Positive |
| 1 × LOD_rep 17 | 24.74 | 80.19 | SARS-CoV-2 | Positive |
| 1 × LOD_rep 18 | 24.95 | 80.22 | SARS-CoV-2 | Positive |
| 1 × LOD_rep 19 | 24.96 | 80.23 | SARS-CoV-2 | Positive |
| 1 × LOD_rep 20 | 25.07 | 80.19 | SARS-CoV-2 | Positive |
| NasoP_rep 1 | 27.88 | 77.64 | Undetermined | Negative |
| NasoP_rep 2 | 28.13 | 77.56, 82.58 | Undetermined | Negative |
| NasoP_rep 3 | 28.76 | 77.55, 82.65 | Undetermined | Negative |
| NasoP_rep 4 | 28.37 | 77.67, 82.22 | Undetermined | Negative |
| NasoP_rep 5 | 28.09 | 82.47 | Undetermined | Negative |
| NasoP_rep 6 | 25.34 | 82.54, 88.49 | Undetermined | Negative |
| NasoP_rep 7 | 27.09 | 82.60, 87.80, | Undetermined | Negative |
| | | 88.52 | | |
| NasoP_rep 8 | 25.55 | 82.54 | Undetermined | Negative |
| NasoP_rep 9 | 26.03 | 82.23, 87.62 | Undetermined | Negative |
| NasoP_rep 10 | 26.35 | 82.45, 88.82 | Undetermined | Negative |

1 × LOD 1.5 × 10³ TCID50/mL

Specificity/Clinical Evaluation

Specificity is the extent to which the method can determine a particular analyte in the analysed matrices without interference from matrix components.

To determine this, RNA was extracted from thirty (30) contrived reactive clinical specimens (virus-spiked) and thirty (30) non-reactive specimens. Twenty of the contrived reactive samples were spiked with the inactivated SARS-CoV-2 virus at 1-2×LOD, while the remaining ten samples will be spiked at varying concentrations spanning the assay testing range. For samples spiked with 1-2×LOD, an acceptance criterion is 95% agreement. For all other concentrations and un-spiked samples, an acceptance criterion is 100% agreement.

TABLE 7
Clinical evaluation with Nasopharyngeal samples

| SARS-CoV-2 | Acceptance | Samples | Direction | |
|---|---|---|---|---|
| Concentration | Criteria | (n) | rate | Result |
| 1 × LOD | 1-2X LOD | 10 | 10/10 | Pass |
| 2 × LOD | 95% agreement | 10 | 10/10 | Pass |
| 10 × LOD | Other | 2 | 2/2 | Pass |
| 17 × LOD | Concentrations | 2 | 2/2 | Pass |
| 33 × LOD | 100% agreement | 2 | 2/2 | Pass |
| 50 × LOD | | 2 | 2/2 | Pass |
| 67 × LOD | | 2 | 2/2 | Pass |
| Unspiked | | 30 | 0/30 | Pass |

Data for the samples follows:

TABLE 8
LOD verification results for nasopharyngeal matrix
spiked with various concentrations of SARS- CoV-2

| | | Melting temp | | |
|---|---|---|---|---|
| Sample name | Ct | (Tm) | Genotype | Result |
| Matrix 1 | 22.285 | 80.37 | SARS-CoV-2 | Positive |
| (1.5 × 10⁴ TCID50/mL) | | | | |
| Matrix 2 | 20.336 | 80.14 | SARS-CoV-2 | Positive |
| (2.5 × 10⁴ TCID50/mL) | | | | |
| Matrix 3 | 21.658 | 80.1 | SARS-CoV-2 | Positive |
| (5.0 × 10⁴ TCID50/mL) | | | | |
| Matrix 4 | 20.858 | 80.29 | SARS-CoV-2 | Positive |
| (7.5 × 10⁴ TCID50/mL) | | | | |
| Matrix 5 | 20.481 | 80.35 | SARS-CoV-2 | Positive |
| (1.0 × 10⁵ TCID50/mL) | | | | |
| Matrix 6 1XLOD* | 24.331 | 80.14 | SARS-CoV-2 | Positive |
| Matrix 7 1XLOD | 25.333 | 80.06 | SARS-CoV-2 | Positive |
| Matrix 8 1XLOD | 26.255 | 80.2 | SARS-CoV-2 | Positive |
| Matrix 9 1XLOD | 24.388 | 80.04 | SARS-CoV-2 | Positive |
| Matrix 10 1XLOD | 25.984 | 80.12 | SARS-CoV-2 | Positive |
| Matrix 11 | 23.293 | 80.05 | SARS-CoV-2 | Positive |
| (1.5 × 10⁴ TCID50/mL) | | | | |
| Matrix 12 | 22.934 | 80.07 | SARS-CoV-2 | Positive |
| (2.5 × 10⁴ TCID50/mL) | | | | |
| Matrix 13 | 21.847 | 80.02 | SARS-CoV-2 | Positive |
| (5.0 × 10⁴ TCID50/mL) | | | | |
| Matrix 14 | 21.187 | 80.03 | SARS-CoV-2 | Positive |
| (7.5 × 10⁴ TCID50/mL) | | | | |
| Matrix 15 | 21.042 | 80.01 | SARS-CoV-2 | Positive |
| (1.0 × 10⁵ TCID50/mL) | | | | |
| Matrix 16 1XLOD | 26.093 | 80.23 | SARS-CoV-2 | Positive |
| Matrix 17 1XLOD | 24.101 | 80.15 | SARS-CoV-2 | Positive |
| Matrix 18 1XLOD | 25.049 | 79.99 | SARS-CoV-2 | Positive |
| Matrix 19 1XLOD | 26.094 | 80.11 | SARS-CoV-2 | Positive |
| Matrix 20 1XLOD | 24.100 | 80.18 | SARS-CoV-2 | Positive |
| Matrix 21 2XLOD# | 24.773 | 80.14 | SARS-CoV-2 | Positive |
| Matrix 22 2XLOD | 25.304 | 80.2 | SARS-CoV-2 | Positive |
| Matrix 23 2XLOD | 23.703 | 80.22 | SARS-CoV-2 | Positive |
| Matrix 24 2XLOD | 25.187 | 80.11 | SARS-CoV-2 | Positive |
| Matrix 25 2XLOD | 240344 | 80.19 | SARS-CoV-2 | Positive |
| Matrix 26 2XLOD | 25.642 | 80.08 | SARS-CoV-2 | Positive |
| Matrix 27 2XLOD | 25.499 | 80.18 | SARS-CoV-2 | Positive |
| Matrix 28 2XLOD | 27.274 | 80.14 | SARS-CoV-2 | Positive |
| Matrix 29 2XLOD | 27.532 | 80.1 | SARS-CoV-2 | Positive |
| Matrix 30 2XLOD | 25.940 | 80.03 | SARS-CoV-2 | Positive |
| Unspiked Matrix 1 | 30.131 | 77.94 | Undermined | Negative |
| Unspiked Matrix 2 | 29.204 | 78.04 | Undermined | Negative |
| Unspiked Matrix 3 | 26.189 | 78.81 | Undermined | Negative |
| Unspiked Matrix 4 | 27.453 | 78.07 | Undermined | Negative |
| Unspiked Matrix 5 | 30.185 | 77.87, 88.23 | Undermined | Negative |
| Unspiked Matrix 6 | 28.678 | 77.99 | Undermined | Negative |
| Unspiked Matrix 7 | 28.610 | 78.04, 87.45, 88.66 | Undermined | Negative |
| Unspiked Matrix 8 | 29.717 | 77.89 | Undermined | Negative |
| Unspiked Matrix 9 | 30.402 | 77.83, 88.22 | Undermined | Negative |
| Unspiked Matrix 10 | 28.287 | 77.99 | Undermined | Negative |
| Unspiked Matrix 11 | 28.537 | NA | Undermined | Negative |
| Unspiked Matrix 12 | 29.777 | 77.89, 85.99, 88.82 | Undermined | Negative |
| Unspiked Matrix 13 | 30.787 | 77.83, 80.15, 86.06 | Undermined | Negative |
| Unspiked Matrix 14 | 28.761 | 77.67, 88.25 | Undermined | Negative |
| Unspiked Matrix 15 | 30.899 | 77.74, 87.05 | Undermined | Negative |
| Unspiked Matrix 16 | 30.281 | 77.75 | Undermined | Negative |
| Unspiked Matrix 17 | 27.530 | 77.79 | Undermined | Negative |
| Unspiked Matrix 18 | 28.022 | 77.96, 87.53 | Undermined | Negative |
| Unspiked Matrix 19 | 29.436 | 77.77 | Undermined | Negative |
| Unspiked Matrix 20 | 26.594 | 80.03 | Undermined | Negative |
| Unspiked Matrix 21 | 28.485 | 77.86 | Undermined | Negative |
| Unspiked Matrix 22 | Undetermined | 77.58 | Undermined | Negative |
| Unspiked Matrix 23 | 26.082 | 78.51 | Undermined | Negative |
| Unspiked Matrix 24 | 28.248 | 77.9 | Undermined | Negative |
| Unspiked Matrix 25 | 27.388 | 77.70, 88.66 | Undermined | Negative |
| Unspiked Matrix 26 | 30.254 | 77.9 | Undermined | Negative |
| Unspiked Matrix 27 | 30.186 | 77.91 | Undermined | Negative |
| Unspiked Matrix 28 | 30.102 | 77.92, 80.09 | Undermined | Negative |
| Unspiked Matrix 29 | 30.659 | 77.87 | Undermined | Negative |
| Unspiked Matrix 30 | 29.990 | 77.95 | Undermined | Negative |

1xLOD (1.5 × 10³ TCID₅₀/mL)
#2xLOD (3.0 × 10³ TCID₅₀/mL)

This validation study demonstrates that the One-step RT-PCR assay, referred to hereinafter as InfectID-COVID-19-R, used with the Mic qPCR hardware (Bio Molecular systems) is suitable for detecting SAR-CoV-2 virus in clinical nasal swabs, with a Limit of Detection established at 1.5×10³ TCID₅₀/mL.

### Example 3 Verification of Test on Other RT-PCR Platforms

Samples tested include: cultured SARS-CoV-2 virus RNA; COVID patient RNA and synthetic oligonucleotide positive controls. These experiments demonstrate that the test is open-platform, and can be used on any real-time PCR platform that has melt-curve capability.

Patient samples tested on the Bio-Rad CFX96 real-time PCR machine.

Expected melt curve temperatures for InfectID-COVID-19-R testing on the Bio-Rad CFX96 machine

| | | Melting |
|---|---|---|
| | | temperature |
| Assay | Target | (tm) |
| InfectID-COVID-19-R | SARS-CoV-2 replication- | 79.5° C. +/− 0.6° C. |
| (Replicating) | competent RNA genome | |

Experimental results (FIG. 7) show the correct melting temperature of 79.5° C. on the BioRad CFX96 machine, which is the expected melting temperature for the InfectID-COVID-19-R test.

Patient Samples Tested on the MIC (Biomolecular Systems) Real-Time PCR Machine.

Expected melt curve temperatures for InfectID-COVID-19-D testing on the MIC machine

| | | MELTING |
|---|---|---|
| | | TEMPERATURE |
| ASSAY | TARGET | (TM) |
| INFECTID-COVID-19- | SARS-CoV-2 | 80.4° C. +/− 1.0° C. |
| R(REPLICATING) | replication- | |
| | competent RNA genome | |

Experimental results (FIG. 8) show the correct melting temperature of 80.47° C. on the MIC machine, which is the expected melting temperature for the InfectID-COVID-19-R test

Patient Samples Tested on the QuantStudio 5 (Applied Biosystems) Real-Time PCR Machine.

Expected melt curve temperatures for InfectID-COVID-19-R testing on the QuantStudio 5 machine

| | | METLTING |
|---|---|---|
| | | TEMPERATURE |
| ASSAY | TARGET | (TM) |
| INFECTID-COVID-19-R | SARS-CoV-2 | 79.8° C. +/0.6° C. |
| (REPLICATING) | replication- | |
| | competent RNA genome | |

Experimental results (FIG. 9) show the correct melting temperature of 79.8° C. on the QuantStudio 5 machine, which is the expected melting temperature for the InfectID-COVID-19-R test.

Patient Samples Tested on the RotorGeneQ (Qiagen) Real-Time PCR Machine.

Expected melt curve temperatures for InfectID-COVID-19-R testing on the RotorGeneQ machine

| | | MELTING |
|---|---|---|
| | | TEMPERATURE |
| ASSAY | TARGET | (TM) |
| INFECTID-COVID-19-R | SARS-CoV-2 | 80.9° C. +/1.0° C. |
| (REPLICATING) | replication- | |
| | competent RNA genome | |

Experimental results (FIG. 10) show the correct melting temperature of 80.41° C. on the RotorGeneQ machine, which is the expected melting temperature for the InfectID-COVID-19-R test.

### Example 4

The InfectID-COVID-19-R assay was validated for 105 SARS CoV-2 positive samples and 105 SARS CoV-2 negative samples against CE IVD certified kits using kit comparison procedure under NABL ISO 15189-2012 guidelines strictly adhering to ICMR protocols. The assay was performed on a RotorGeneQ RT-PCR machine and a positive test showed the expected melting temperature set forth in example 3 for that machine. The kit used for nucleic acid extraction were Qiagen viral RNA Nucleic acid extraction. PCR was performed for extracted RNAs using RealStar SARS-CoV-2 rt-pcr Kit 1.0 for comparison.

Results were as follows:

| | RT-PCR test (RealStar | | |
|---|---|---|---|
| | *SARS-CoV-2 rt-pcr Kit) | | |
| InfectID-COVID-19-R | Positive | Negative | Total |
| Positive | 97(TP) | 5(FP) | |
| Negative | 8(FN) | 100(TN) | |
| Total | 105 | 105 | 210 |

TP - True positive
TN-True negative
FP-False positive
FN- False Negative
Sensitivity: TP/(TP + FN) = 97/(97 + 8) = 92.38%
Specificity: TN/(TN + FP) = 100/(100 + 5) = 95.23%
Positive Predicative Value: TP/(TP + FP) = 97/(97 + 5) = 95.09%
Negative Predictive Value: TN/(TN + FN) = 100/100 + 8) = 92.59%

The test was also compared to applicant's validated test for identification of covid-19, InfectID-COVID-19-D (described in Australian provisional application No. 2020902627 filed on 27 Jul. 2020, the contents of which are incorporated herein by reference), and against RealStar*SARS-CoV-2 rt-pcr Kit 1.0 to trace the progression ofthe disease for 12 samples.

Sample 1

| Kit | Day 1 | Day 2 | Day 3 | Day 4 | Day 5 | Day 6 | Day 7 | Day 8 | Day 9 | Day 10 | Day 11 | Day 12 |
|---|---|---|---|---|---|---|---|---|---|---|---|---|
| InfectID | Pos | Pos | Pos | Pos | Pos | Neg | Neg | Neg | Neg | Neg | Neg | Neg |
| COVID-R | | | | | | | | | | | | |
| InfectID | Pos | Pos | Pos | Pos | Pos | Pos | Pos | Pos | Neg | Neg | Neg | Neg |
| COVID-D | | | | | | | | | | | | |
| RealStar*SARS- | Pos | Pos | Pos | Pos | Pos | Pos | Pos | Pos | Neg | Neg | Neg | Neg |
| CoV-2 rt-pcr | | | | | | | | | | | | |
| Kit 1.0 | | | | | | | | | | | | |

Sample 2

| Kit | Day 1 | Day 2 | Day 3 | Day 4 | Day 5 | Day 6 | Day 7 | Day 8 | Day 9 | Day 10 | Day 11 | Day 12 |
|---|---|---|---|---|---|---|---|---|---|---|---|---|
| InfectID | Pos | Pos | Pos | Pos | Pos | Pos | Pos | Neg | Neg | Neg | Neg | Neg |
| COVID-R | | | | | | | | | | | | |
| InfectID | Pos | Pos | Pos | Pos | Pos | Pos | Neg | Neg | Pos | Neg | Neg | Neg |
| COVID-D | | | | | | | | | | | | |
| RealStar*SARS- | Pos | Pos | Pos | Pos | Pos | Pos | Pos | Pos | Neg | Neg | Neg | Neg |
| CoV-2 rt-pcr | | | | | | | | | | | | |
| Kit 1.0 | | | | | | | | | | | | |

Sample 3

| Kit | Day 1 | Day 2 | Day 3 | Day 4 | Day 5 | Day 6 | Day 7 | Day 8 | Day 9 | Day 10 | Day 11 | Day 12 |
|---|---|---|---|---|---|---|---|---|---|---|---|---|
| InfectID | Pos | Pos | Pos | Pos | Pos | Pos | Neg | Pos | Neg | Neg | Neg | Neg |
| COVID-R | | | | | | | | | | | | |
| InfectID | Pos | Pos | Pos | Pos | Pos | Pos | Pos | Neg | Neg | Neg | Neg | Neg |
| COVID-D | | | | | | | | | | | | |
| RealStar*SARS- | Pos | Pos | Pos | Pos | Pos | Pos | Pos | Pos | Neg | Neg | Neg | Neg |
| CoV-2 rt-pcr | | | | | | | | | | | | |
| Kit 1.0 | | | | | | | | | | | | |

Sample 4

| Kit | Day 1 | Day 2 | Day 3 | Day 4 | Day 5 | Day 6 | Day 7 | Day 8 | Day 9 | Day 10 | Day 11 | Day 12 |
|---|---|---|---|---|---|---|---|---|---|---|---|---|
| InfectID | Pos | Pos | Pos | Pos | Pos | Neg | Neg | Neg | Pos | Neg | Neg | Neg |
| COVID-R | | | | | | | | | | | | |
| InfectID | Pos | Pos | Pos | Pos | Pos | Pos | Pos | Pos | Neg | Neg | Neg | Neg |
| COVID-D | | | | | | | | | | | | |
| RealStar*SARS- | Pos | Pos | Pos | Pos | Pos | Pos | Neg | Neg | Neg | Neg | Neg | Neg |
| CoV-2 rt-pcr | | | | | | | | | | | | |
| Kit 1.0 | | | | | | | | | | | | |

Sample 5

| Kit | Day 1 | Day 2 | Day 3 | Day 4 | Day 5 | Day 6 | Day 7 | Day 8 | Day 9 | Day 10 | Day 11 | Day 12 |
|---|---|---|---|---|---|---|---|---|---|---|---|---|
| InfectID | Pos | Pos | Pos | Pos | Pos | Pos | Pos | Neg | Neg | Neg | Neg | Neg |
| COVID-R | | | | | | | | | | | | |
| InfectID | Pos | Pos | Pos | Pos | Pos | Pos | Pos | Pos | Neg | Neg | Neg | Neg |
| COVID-D | | | | | | | | | | | | |
| RealStar*SARS- | Pos | Pos | Pos | Pos | Pos | Pos | Pos | Neg | Neg | Neg | Neg | Neg |
| CoV-2 rt-pcr | | | | | | | | | | | | |
| Kit 1.0 | | | | | | | | | | | | |

Sample 6

| Kit | Day 1 | Day 2 | Day 3 | Day 4 | Day 5 | Day 6 | Day 7 | Day 8 | Day 9 | Day 10 | Day 11 | Day 12 |
|---|---|---|---|---|---|---|---|---|---|---|---|---|
| InfectID | Pos | Pos | Pos | Pos | Pos | Pos | Pos | Pos | Neg | Neg | Neg | Neg |
| COVID-R | | | | | | | | | | | | |
| InfectID | Pos | Pos | Pos | Pos | Pos | Pos | Pos | Pos | Neg | Neg | Neg | Neg |
| COVID-D | | | | | | | | | | | | |
| RealStar*SARS- | Pos | Pos | Pos | Pos | Pos | Pos | Pos | Pos | Neg | Neg | Neg | Neg |
| CoV-2 rt-pcr | | | | | | | | | | | | |
| Kit 1.0 | | | | | | | | | | | | |

Sample 7

| Kit | Day 1 | Day 2 | Day 3 | Day 4 | Day 5 | Day 6 | Day 7 | Day 8 | Day 9 | Day 10 | Day 11 | Day 12 |
|---|---|---|---|---|---|---|---|---|---|---|---|---|
| InfectID | Pos | Pos | Pos | Pos | Pos | Pos | Pos | Neg | Neg | Neg | Neg | Neg |
| COVID-R | | | | | | | | | | | | |
| InfectID | Pos | Pos | Pos | Pos | Pos | Pos | Pos | Neg | Neg | Neg | Neg | Neg |
| COVID-D | | | | | | | | | | | | |
| RealStar*SARS- | Pos | Pos | Pos | Pos | Pos | Pos | Neg | Neg | Neg | Neg | Neg | Neg |
| CoV-2 rt-pcr | | | | | | | | | | | | |
| Kit 1.0 | | | | | | | | | | | | |

Sample 8

| Kit | Day 1 | Day 2 | Day 3 | Day 4 | Day 5 | Day 6 | Day 7 | Day 8 | Day 9 | Day 10 | Day 11 | Day 12 |
|---|---|---|---|---|---|---|---|---|---|---|---|---|
| InfectID | Pos | Pos | Pos | Pos | Pos | Pos | Pos | Pos | Pos | Neg | Neg | Neg |
| COVID-R | | | | | | | | | | | | |
| InfectID | Pos | Pos | Pos | Pos | Pos | Pos | Pos | Neg | Neg | Neg | Neg | Neg |
| COVID-D | | | | | | | | | | | | |
| RealStar*SARS- | Pos | Pos | Pos | Pos | Pos | Pos | Pos | Neg | Neg | Neg | Neg | Neg |
| CoV-2 rt-pcr | | | | | | | | | | | | |
| Kit 1.0 | | | | | | | | | | | | |

Sample 9

| Kit | Day 1 | Day 2 | Day 3 | Day 4 | Day 5 | Day 6 | Day 7 | Day 8 | Day 9 | Day 10 | Day 11 | Day 12 |
|---|---|---|---|---|---|---|---|---|---|---|---|---|
| InfectID | Pos | Pos | Pos | Pos | Pos | Pos | Neg | Pos | Neg | Neg | Neg | Neg |
| COVID-R | | | | | | | | | | | | |
| InfectID | Pos | Pos | Pos | Pos | Pos | Pos | Pos | Neg | Neg | Neg | Neg | Neg |
| COVID-D | | | | | | | | | | | | |
| RealStar*SARS- | Pos | Pos | Pos | Pos | Pos | Pos | Pos | Pos | Neg | Neg | Neg | Neg |
| CoV-2 rt-pcr | | | | | | | | | | | | |
| Kit 1.0 | | | | | | | | | | | | |

Sample 10

| Kit | Day 1 | Day 2 | Day 3 | Day 4 | Day 5 | Day 6 | Day 7 | Day 8 | Day 9 | Day 10 | Day 11 | Day 12 |
|---|---|---|---|---|---|---|---|---|---|---|---|---|
| InfectID | Pos | Pos | Pos | Pos | Pos | Pos | Pos | Neg | Neg | Neg | Neg | Neg |
| COVID-R | | | | | | | | | | | | |
| InfectID | Pos | Pos | Pos | Pos | Pos | Pos | Pos | Neg | Neg | Neg | Neg | Neg |
| COVID-D | | | | | | | | | | | | |
| RealStar*SARS- | Pos | Pos | Pos | Pos | Pos | Pos | Pos | Neg | Neg | Neg | Neg | Neg |
| CoV-2 rt-pcr | | | | | | | | | | | | |
| Kit 1.0 | | | | | | | | | | | | |

Sample 11

| Kit | Day 1 | Day 2 | Day 3 | Day 4 | Day 5 | Day 6 | Day 7 | Day 8 | Day 9 | Day 10 | Day 11 | Day 12 |
|---|---|---|---|---|---|---|---|---|---|---|---|---|
| InfectID | Pos | Pos | Pos | Pos | Pos | Pos | Neg | Pos | Neg | Neg | Neg | Neg |
| COVID-R | | | | | | | | | | | | |
| InfectID | Pos | Pos | Pos | Pos | Pos | Pos | Neg | Pos | Neg | Neg | Neg | Neg |
| COVID-D | | | | | | | | | | | | |
| RealStar*SARS- | Pos | Pos | Pos | Pos | Pos | Pos | Neg | Neg | Neg | Neg | Neg | Neg |
| CoV-2 rt-pcr | | | | | | | | | | | | |
| Kit 1.0 | | | | | | | | | | | | |

Sample 12

| Kit | Day 1 | Day 2 | Day 3 | Day 4 | Day 5 | Day 6 | Day 7 | Day 8 | Day 9 | Day 10 | Day 11 | Day 12 |
|---|---|---|---|---|---|---|---|---|---|---|---|---|
| InfectID | Pos | Pos | Pos | Pos | Pos | Pos | Pos | Neg | Neg | Pos | Neg | Neg |
| COVID-R | | | | | | | | | | | | |
| InfectID | Pos | Pos | Pos | Pos | Pos | Pos | Pos | Neg | Neg | Pos | Neg | Neg |
| COVID-D | | | | | | | | | | | | |
| RealStar*SARS- | Pos | Pos | Pos | Pos | Pos | Pos | Pos | Neg | Neg | Neg | Neg | Neg |
| CoV-2 rt-pcr | | | | | | | | | | | | |
| Kit 1.0 | | | | | | | | | | | | |

The samples show that replicating virus may not be present even when a patient sample still tests positive to Covid-19.

1. A method for detecting replication of severe acute respiratory syndrome coronavirus 2 (SARS-CoV-2) in a sample, said method including the steps of:
(a) amplifying a target nucleic acid from a portion of subgenomic mRNA of SARS-CoV-2 from the sample; and
(b) detecting the presence or absence of the target nucleic acid amplified in step (a).

2. The method of claim 1, wherein the presence or absence of the target nucleic acid is detected at least in part using high resolution melt analysis.

3. The method of claim 1, including the further step of detecting the presence or absence of SARS-CoV-2 genomic RNA in the sample.

4. The method of claim 1, wherein the sample is a biological sample, such as cells, blood, serum, plasma, saliva, cerebrospinal fluid, urine, stool, sputum, nasopharyngeal aspirates or swabs, obtained from a subject.

5. A method of determining a prognosis for a SARS-CoV-2 infection in a subject, said method including the steps of:
(a) amplifying a target nucleic acid from a portion of subgenomic mRNA of SARS-CoV-2 from a biological sample from the subject; and
(b) detecting the presence or absence of the target nucleic acid amplified in step (a), wherein the presence of absence of the target nucleic acid indicates a more or less favourable prognosis for the SARS-CoV-2 infection in the subject.

6. The method of claim 5, wherein step (b) further includes measuring an expression level of the target nucleic acid, wherein the expression level of the target nucleic acid indicates or correlates with a level of replication of SARS-CoV-2 in the biological sample.

7. The method of claim 6, a high or increased expression level of the target nucleic acid and/or level of replication can indicate or correlate with a less favourable prognosis for the SARS-CoV-2 infection and/or a low or decreased expression level of the target nucleic acid and/or level of replication can indicate or correlate with a more favourable prognosis for the SARS-CoV-2 infection.

8. A method for identifying an agent capable of altering or modulating replication of SARS-CoV-2 in one or more cells infected therewith, said method including the steps of:
(a) amplifying a target nucleic acid from a portion of subgenomic mRNA of SARS-CoV-2 from the one or more cells treated with the agent; and
(b) measuring an expression level of the target nucleic acid amplified in step (a), wherein the expression level of the target nucleic acid indicates or correlates with a level of replication of SARS-CoV-2 in the one or more cells; and
wherein:
the level of replication of: (i) a first population of the one or more cells treated with the agent; and (ii) a second population of the one or more cells not treated with the agent is determined; and
the method further comprising comparing the level of replication of the first and second population of cells, wherein an alteration or modulation of the level of replication determined for the first and second populations of the cells indicates that the agent is capable of altering replication of SARS-CoV-2.

9. (canceled)

10. The method of claim 8, comprising determining the level of replication of the cells at respective first and second time points; and comparing the level of replication of the cells at the first and second time points, wherein an alteration or modulation of the level of replication measured at the first and second time points indicates that the agent is capable of altering replication of SARS-CoV-2.

11. The method of claim 8, further including the initial steps of infecting the one or more cells with SARS-CoV-2 and/or treating the one or more cells with the agent.

12. The method of claim 1, wherein the portion of subgenomic mRNA comprises at least a portion of a leader sequence.

13. The method of claim 1, wherein the portion of subgenomic mRNA comprises at least a portion of an E gene or gene product.

14. The method of claim 1, wherein the portion of subgenomic mRNA of SARS-CoV-2 comprises, consists of or consists essentially of a nucleotide sequence set forth in SEQ ID NOs:6.

15. The method of claim 1, wherein amplifying the target nucleic acid includes using a forward primer comprising, consisting of or consisting essentially of a nucleotide sequence set forth in SEQ ID NO:4, a nucleotide sequence complementary thereto or a fragment or variant thereof and/or a reverse primer comprising, consisting of or consisting essentially of a nucleotide sequence set forth in SEQ ID NO:5, a nucleotide sequence complementary thereto or a fragment or variant thereof.

16. The method of claim 1, wherein amplifying the target nucleic acid utilizes reverse transcriptase-polymerase chain reaction (RT-PCR).

17. The method of claim 15, further including the steps of obtaining RNA from the sample, the biological sample or one or more cells and reverse transcribing the RNA to obtain cDNA.

18. An isolated oligonucleotide or primer comprising, consisting of or consisting essentially of a nucleic acid sequence as set forth in any one of SEQ ID NOs:4 and 5, a nucleotide sequence complementary thereto or a fragment or variant thereof.

19. An array comprising the isolated oligonucleotide of claim 18.

20. A biochip comprising a solid substrate and the isolated oligonucleotide of claim 18.

21. A kit or assay for detecting replication of SARS-CoV-2 in a sample, said kit or assay comprising: the isolated oligonucleotide of claim 17.

22. The kit or assay of claim 21, comprising a pair of oligonucleotides, wherein at least one of the pair of oligonucleotides comprises, consists of or consists essentially of the nucleic acid sequence as set forth in any one of SEQ ID NOs:4 and 5 and, optionally,
further comprising a second pair of primers for detecting the presence or absence of SARS-CoV-2 genomic RNA.

23.-24. (canceled)


SEQUENCE LISTING (6 sequences)

SEQ ID NO: 1 - DNA, 19841 bp, Wuhan SARS-CoV-2 strain
atggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcactaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgataaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtcggaggtaaaggcttttgcaaactacacaattggaattgtgttaattgtgatacattctgtgctggtagtacatttattagtgatgaagttgcgagagacttgtcactacagtttaaaagaccaataaatcctttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgcttttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaaagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgcagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattgcttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaa

SEQ ID NO: 2 - DNA, 24 bp, Artificial Sequence
  Primer
cttggctattcagttgttgctatc

SEQ ID NO: 3 - DNA, 24 bp, Artificial sequence
  Primer
gtgagatggatccgagacaataat

SEQ ID NO: 4 - DNA, 23 bp, Artificial Sequence
  Primer
cgatctcttgtagatctgttctc

SEQ ID NO: 5 - DNA, 22 bp, Artificial Sequence
  Primer
atattgcagcagtacgcacaca

SEQ ID NO: 6 - DNA, 171 bp, Artificial Sequence
  Target nucleic acid amplicon.
cgatctcttgtagatctgttctctaaacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatat